(19) United States
(12) Patent Application Publication
(10) Pub. No.: US 2025/0380902 A1
(43) Pub. Date: Dec. 18, 2025

(54) REAL-TIME EARLY STAGE DELIRIUM DETECTION AND MANAGEMENT

(71) Applicant: PASCALL Systems, Inc., Boston, MA (US)

(72) Inventors: Patrick Purdon, Portola Valley, CA (US); Tuan Le Mau, Boston, MA (US); Yao Zhao, Brookline, MA (US)

(73) Assignee: PASCALL Systems, Inc., Boston, MA (US)

(21) Appl. No.: 19/229,577

(22) Filed: Jun. 5, 2025

Related U.S. Application Data
(60) Provisional application No. 63/656,186, filed on Jun. 5, 2024.
(60) Provisional application No. 63/684,170, filed on Aug. 16, 2024.
(60) Provisional application No. 63/690,086, filed on Sep. 3, 2024.
(60) Provisional application No. 63/701,697, filed on Oct. 1, 2024.

(51) Int. Cl. A61B 5/00; A61B 5/291; A61B 5/31; A61B 5/374; A61B 5/375

(52) U.S. Cl. CPC A61B 5/4821; A61B 5/291; A61B 5/31; A61B 5/374; A61B 5/375; A61B 5/4839; A61B 5/7253; A61B 5/746

(57) ABSTRACT

Disclosed are systems and methods that provide a novel computerized framework for a closed-loop, decision-intelligence (DI)-based computerized framework for automatically and dynamically managing and controlling a medical procedure, inclusive of administered medication and/or anesthesia to a patient and an intraoperative level of consciousness of the patient. The disclosed framework provides an improved electroencephalography (EEG) indices that adapts to specific patient needs, and dynamically adapts to factors of an ongoing procedure to ensure that the proper levels of anesthesia are administered, required and/or maintained. This provides computerized capabilities to maintain safe levels of the patient's consciousness, such that post-operative patient health is preserved and maintained. Thus, the disclosed framework provides an effective anesthesia management framework that can be leveraged to safely manage a patient's health during and after a medical procedure for which anesthesia is used.

### CROSS-REFERENCE TO RELATED APPLICATIONS

This application claims the benefit of and priority to U.S. Provisional Application No. 63/656,186, filed Jun. 5, 2024, U.S. Provisional Application No. 63/684,170, filed Aug. 16, 2024, U.S. Provisional Application No. 63/690,086, filed Sep. 3, 2024 and U.S. Provisional Application No. 63/701,697, filed Oct. 1, 2024, each of which are incorporated herein by reference in their entirety.

### FIELD OF THE DISCLOSURE

The present disclosure provides a decision intelligence (DI)-based computerized framework for automatically and dynamically managing and controlling a medical procedure and a patient during such procedure, inclusive of the administration of medication and/or anesthesia, and monitoring of a patient based therefrom during and/or after such procedure.

### GOVERNMENT CONTRACT

Not applicable.

### STATEMENT RE: FEDERALLY FUNDED SPONSORED RESEARCH/DEVELOPMENT

SUPPORTED BY FEDERAL GRANT AG066325 AWARDED TO PASCALL SYSTEMS, INC.

### BACKGROUND

Anesthesia for surgery is a medical procedure that involves using medications to induce a temporary loss of sensation or consciousness in order to perform surgical procedures without causing pain or discomfort.

### SUMMARY OF THE DISCLOSURE

Each day, more than 100,000 patients undergo general anesthesia in the United States. 40 percent are 60 years or older. Post-operative delirium (POD) in these patients have been recognized as a major problem since at least the 1950s. POD is a transient, acute state of confusion that is associated with increased hospital length of stay, increased likelihood of subsequent cognitive problems, and increased mortality that can occur in up to 47% of patients. Older patients with pre-existing or un-diagnosed Alzheimer's Disease and Related dementias (ADRD) are at greater risk of developing POD. Patients who develop POD have a very poor prognosis: they have ˜50% longer length of stay, are more likely to be discharged to a skilled nursing facility have 2- to 3-fold higher one year mortality, and 7-fold higher five year mortality. Patients who develop POD also experience significant long-term functional and cognitive decline comparable in magnitude to mild cognitive impairment, with 3 to 4 fold higher rates of cognitive decline many years after surgery compared to patients without delirium. Patients with ADRD are at greater risk of developing POD and have significantly worse cognitive outcomes after delirium compared to patients without ADRD. Patients who experience POD incur ˜$44,000 for Medicare per patient per year and $32 billion nationwide.

Once delirium sets in, treatment options are limited. Family members often feel powerless, witnessing their loved ones undergo repeated cycles of sedation, antipsychotic medication, physical restraints, and distress as their condition worsens. However, many cases of delirium are preventable. Intensive clinical intervention programs such as the Hospital Elder Life Program (HELP) can reduce POD incidence by 35% or more by providing a holistic operative care targeting POD risk factors such as pre-existing brain health, sensory and sleep deprivation, dehydration. HELP is labor intensive and expensive, requiring a team consisting of a geriatrician, geriatric nurse specialist, two elder life specialists, therapeutic recreation specialist, physical therapist, and trained volunteers to implement. Alternatively, reducing excessive anesthetic exposure can significantly prevent POD with the advantage of being labor-efficient and scalable. First, anesthetic drugs can harm the brain directly, causing issues like increased beta-amyloid accumulation and tau phosphorylation. Secondly, anesthetics can induce systemic hypotension and in older patients could elevate the risk of POD. EEG-guided anesthetic management, a major recommendation by the Perioperative Brain Health Initiative (PBHI-ASA) in 2018, can help minimize anesthetic exposure.

Existing efforts to use EEG monitors, particularly the market-leading Medtronic Bispectral Index (BIS) (70% market share) and Massimo SEDLINE (20% market share) to provide EEG-guided anesthetic management has shown mixed results in preventing POD. Several high quality randomized controlled trials overall show a modest net reduction averaging 4% from an initial incidence baseline of 25%. This modest outcome can be attributed in part to the limitations of existing anesthetic brain monitors like BIS, which was last updated in 2005. The BIS was developed using data from young patients and is highly inaccurate in older patients who are at highest risk of POD, often providing higher values than appropriate, leading anesthesiologists to place elderly and ADRD patients in burst suppression, a deep anesthetic brain state that is linked to increased risk of POD. Furthermore, elderly patients at elevated risk for conditions like ADRD appear to have lower anesthetic requirements than what is predicted by conventional age-adjusted pharmacodynamic models. Anesthesiologists, guided by readings from BIS or analogous monitors, might hesitate to decrease their anesthetic doses even when it might be appropriate. This reluctance could potentially offset the advantages of EEG-guidance, significantly constraining the reduction in anesthetic exposure. For over 20 years, industry incumbents (i.e. Medtronic BIS) have done little to improve their EEG indices despite numerous reports to the FDA on their reliability. It is therefore not a surprise that anesthesiologists have mixed feelings about using BIS or SEDLINE and that adoption has been limited.

To that end, the disclosed systems and methods provide a computerized framework that addresses existing technical shortcomings, among others, by providing the disclosed M1 index and corresponding functionality and capabilities. In some embodiments, the disclosed systems and methods provide functionality for accurate EEG-guided anesthetic management for aging and ADRD patients at risk of POD, as discussed herein. In some embodiments, the framework provides functionality for anesthesia-induced alpha oscillations that exhibit diminished power in both aging and ADRD patients, suggesting decreased anesthetic needs, as discussed herein.

According to some embodiments, the disclosed systems and methods provide an EEG-based index that accurately monitors anesthetic brain state while at the same time provides information to help minimize risk of POD via intraoperative anesthetic management. The disclosed M1 index, as discussed in more detail below, provides accurate readings whether a patient is young, old, and/or with ADRD. Conversely, the conventional BIS algorithm may work in young patients but shows erroneously high readings in old and ADRD patients. Indeed, as discussed herein, the M1 index can distinguish patients with higher POD risk to guide titration to lower anesthetic exposure, while the BIS algorithm fails to distinguish the two groups of patients. Moreover, a delirium predictor based on the M1 index achieves 100% accuracy in identifying patients at risk of POD, while BIS is unable to do so reliably. By accounting for aging and ADRD patients, M1 could be capable of reducing POD by 50% or more, 3× better than BIS performance.

According to some embodiments, as discussed herein, the administration of medication, particularly anesthesia, during medical procedures is a critical aspect of patient care that requires careful monitoring and management to avoid post-operative complications. Such complications can include, but are not limited to, POD, depression, post-operative cognitive dysfunction (POCD), postoperative nausea and vomiting (PONV), respiratory complications (e.g., atelectasis, pneumonia, respiratory failure), cardiovascular issues (e.g., arrhythmias, myocardial infarction, stroke), chronic postoperative pain syndromes, thromboembolism (e.g., deep vein thrombosis, pulmonary embolism), endocrine and metabolic disturbances, allergic reactions or adverse drug reactions, postoperative fever, postoperative ileus, peripheral nerve injuries, and the like.

Such adverse outcomes can significantly impact a patient's recovery and overall well-being, making it essential for healthcare providers to implement comprehensive strategies to mitigate these risks.

According to some embodiments, as discussed herein, the process begins with a thorough pre-operative assessment, where an evaluation of the patient's medical history, including any pre-existing conditions or medications that might interact with anesthesia. Such evaluation also includes assessing risk factors for post-operative delirium and depression, such as advanced age, history of cognitive impairment, or previous episodes of depression. By identifying these risk factors early, the medical team can tailor their approach to minimize potential complications.

During the procedure itself, advanced monitoring systems can play a crucial role in tracking vital signs and ensuring patient safety. These systems continuously measure and display key physiological parameters such as blood pressure, heart rate, ECG, oxygen saturation, end-tidal CO2, and body temperature. In addition to these standard measurements, specialized monitoring techniques are employed to assess the depth of anesthesia. EEG-based systems like the Bispectral Index (BIS) or Entropy provide real-time information about the patient's level of consciousness, allowing anesthesiologists to fine-tune the administration of anesthetic agents. Neuromuscular blockade is also closely monitored to ensure proper muscle relaxation without excessive paralysis.

As discussed herein, in some embodiments, the management of anesthesia itself is a delicate balance, which via the disclosed systems and methods, can be dynamically determined and applied via a closed-loop system/framework. As discussed herein the disclosed mechanisms allow for careful titration of anesthesia to maintain an appropriate depth while avoiding overdose. In some cases, regional anesthesia techniques may be considered to reduce overall anesthetic requirements and potentially decrease the risk of post-operative cognitive dysfunction.

In some embodiments, the choice of specific medications used during the procedure can have a significant impact on post-operative outcomes. Anesthetic agents that are less likely to cause cognitive dysfunction, such as propofol, may be preferred over alternatives like benzodiazepines. Additionally, healthcare providers aim to avoid anticholinergic medications when possible, as these drugs can increase the risk of delirium. Short-acting agents are often favored for their easier titration and faster recovery profiles, allowing for more precise control over the depth and duration of anesthesia.

Perioperative care can extend beyond just the administration of anesthesia. Maintaining proper oxygenation and ventilation is crucial for preserving cognitive function. Adequate cerebral perfusion must be ensured by carefully managing blood pressure throughout the procedure. Normothermia, or maintaining normal body temperature, is another important factor in reducing the risk of post-operative complications. Effective pain management using multimodal analgesia techniques is also essential, as poorly controlled pain can contribute to both delirium and depression.

Post-operative monitoring can be critical for early detection and intervention of any developing complications. Regular cognitive assessments using tools like the Confusion Assessment Method for ICU (CAM-ICU) can help identify signs of delirium early. Similarly, validated screening tools are employed to monitor for signs of depression in the post-operative period. Continuing to manage pain effectively during recovery is crucial for reducing stress and minimizing the potential for depression.

Accordingly, in some embodiments, as discussed herein, effectively monitoring and managing medication administration to avoid POD and other medical and/or mental health conditions, inter alia, can ensure the best possible care for the patient. Such computerized and DI-based approach extends from pre-operative planning through post-operative care and follow-up.

Moreover, the disclosed systems and methods, which are effectuated via the M1 index and corresponding operational framework discussed herein, can be utilized to control, manage and/or manipulate a level of consciousness of a patient during a procedure and/or while they are under anesthesia.

Under existing mechanisms, maintaining a specific level of consciousness or unconsciousness during a medical procedure involving anesthesia can be challenging due to several factors. One primary issue is achieving the precise balance between anesthesia and consciousness levels, as the effects of anesthetic agents can vary greatly among individuals. Over-sedation may lead to an unnecessary depth of unconsciousness, while under-sedation risks patient discomfort or awareness during the procedure. Additionally, patient-specific variables such as age, weight, and overall health can influence how their body metabolizes anesthesia, complicating the task of fine-tuning dosages. Monitoring and adjusting the anesthetic levels requires constant vigilance and may involve real-time assessments of the patient's vital signs and responses. Furthermore, interactions between anesthetic agents and other medications can alter their effectiveness, potentially leading to unexpected variations in consciousness levels. These complexities necessitate careful management and continuous adjustment to ensure that the patient remains in the intended state of consciousness or unconsciousness throughout the procedure. While maintaining an intended state of consciousness or unconsciousness is challenging on its own, avoiding unnecessary depth of unconsciousness is also challenging if not impossible without appropriate monitoring. That unnecessary depth of unconsciousness is thought to be a major contributing factor to POD, but existing technologies such as BIS or Sedline have not been explicitly designed to indicate both states of consciousness and/or unconsciousness contemporaneously with information about anesthesia-related risk of POD. Furthermore, existing technologies such as BIS or Sedline have not been designed to account for patients' state of cognitive health (e.g., different levels of pre-clinical, clinical, or undiagnosed Alzheimer's Disease or related dementias, or different levels of cognitive impairment that may be diagnosed or undiagnosed), which can significantly influence both the EEG signal and patients' sensitivity to anesthetic drugs. Accordingly, as described earlier, unlike the M1 index described herein, these existing technologies fail to indicate or predict such POD risk.

To that end, the disclosed systems and methods provide a novel functional framework that can leverage the M1 index discussed herein maintain a patient's level of consciousness at safe and desired levels during an operation, such that post-operative patient health can be maintained (e.g., how and/or the manner in which they wake from the medication, and resume normal brain activity, and minimize the risk of POD). Accordingly, as discussed herein, the disclosed framework operates by analyzing EEG signals (e.g., frontal EEG signals) in accordance with the compilation and curation of the M1 index so as to ensure a desired range of EEG signals are maintained (e.g., 0-100, as per conventional EEG products' ranges). Thus, via such EEG signal analytics, the disclosed framework can execute state-space modeling of distinct EEG dynamics as well as artifact dynamics to extract EEG features to ensure that EEG signals are maintained within the defined ranges.

According to some embodiments, a method is disclosed, which includes executable steps for: collecting data about a user, the user data comprising metrics indicative of vitals of the user; analyzing electroencephalography (EEG) signals of the user; determining, based on the EEG analysis, an M1 index, the M1 index providing informational values that correspond to a level of consciousness or unconsciousness of a patient when they are subject to anesthesia, as well as providing informational values that correspond to increasing or decreasing risk for post-operative delirium; managing the EEG signals of the user during a procedure via the M1 index; and managing the level of consciousness and risk of post-operative delirium of the patient based on the managed EEG signals and the M1 index.

In some embodiments, the methods can further include compiling the M1 index prior to the procedure. In some embodiments, the methods can further include compiling the M1 index during the procedure, and updating the M1 index based on real-time analysis of the EEG signals during the procedure. In some embodiments, the methods can further include compiling the M1 index during post-operative awaking of the user. In some embodiments, the methods can further include modifying an amount of anesthesia based on the management of the EEG signals and/or management of the level of consciousness.

According to some embodiments, a system is disclosed, which include a processor configured to, inter alia: collect data about a user, the user data comprising metrics indicative of vitals of the user; analyze electroencephalography (EEG) signals of the user; determine, based on the EEG analysis, an M1 index, the M1 index providing informational values that correspond to a level of consciousness or unconsciousness of a patient when they are subject to anesthesia, as well as providing informational values that correspond to increasing or decreasing risk for post-operative delirium; manage the EEG signals of the user during a procedure via the M1 index; and manage the level of consciousness and risk of post-operative delirium of the patient based on the managed EEG signals and the M1 index.

According to some embodiments, a non-transitory computer-readable storage medium tangibly encoded with computer-executable instructions is disclosed, such that when the instructions are executed by a processor, perform a method including: collecting data about a user, the user data comprising metrics indicative of vitals of the user; analyzing electroencephalography (EEG) signals of the user; determining, based on the EEG analysis, an M1 index, the M1 index providing informational values that correspond to a level of consciousness of a patient when they are subject to anesthesia; managing the EEG signals of the user during a procedure via the M1 index; and managing the level of consciousness or unconsciousness of the user based on the managed EEG signals and the M1 index.

According to some embodiments, a method is disclosed for a closed-loop, DI-based computerized framework for automatically and dynamically managing and controlling a medical procedure, inclusive of administered anesthesia to a patient. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for automatically and dynamically managing and controlling a medical procedure, inclusive of administered anesthesia to a patient.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

According to some embodiments, the disclosed systems and methods can be used to indicate a range of M1 values that would reduce the risk of post-operative delirium related to anesthetic exposure during the maintenance of a level of consciousness or unconsciousness consistent with general anesthesia or sedation. Furthermore, according to some embodiments, the disclosed systems and methods can be used to indicate a range of M1 values that would increase the risk of post-operative delirium during the maintenance of a level of consciousness or unconsciousness consistent with general anesthesia or sedation.

According to some embodiments, as disclosed in APPENDIX A from U.S. Provisional No. 63/701,697, from which this application depends, and is incorporated herein by reference, the disclosed systems and methods can be utilized to perform early stage delirium detection during a medical procedure (e.g., within a first n minutes (e.g., 20 minutes) for example). According to some embodiments, early detection of delirium during medical procedures offers significant benefits for patient care and outcomes. Delirium, characterized by acute confusion and altered consciousness, can occur in patients undergoing various medical interventions, especially in hospital settings. Recognizing delirium early allows for prompt intervention, potentially reducing its severity and duration. This can lead to shorter hospital stays, decreased mortality rates, and improved long-term cognitive outcomes. Early detection also enables healthcare providers to identify and address underlying causes, such as medication side effects, infections, or metabolic imbalances.

Furthermore, early recognition of delirium helps protect patient safety. Delirious patients may attempt to remove medical devices or leave their beds, risking injury or disruption of treatment. Timely detection allows for appropriate supervision and safety measures to be implemented.

Early intervention can also alleviate distress for both patients and their families. Delirium can be a frightening experience, and prompt management can reduce anxiety and improve overall patient comfort. Lastly, early detection of delirium can lead to more efficient resource allocation in healthcare settings. By addressing the condition promptly, complications that might require intensive care or prolonged hospitalization can be minimized, ultimately reducing healthcare costs and improving overall patient flow.

Furthermore, the occurrence of POD is known to increase the risk of many undesirable post-operative outcomes, including increased length of stay, increased healthcare cost, increased risk of cognitive decline, loss of functional independence, discharge to skilled nursing facilities, post-operative cognitive disorder or post-operative neurocognitive disorder, to name a few. Thus, while the discussion herein may exemplify indicators to predict or quantify risk of POD, it can also readily provide indicators of other post-operative outcomes that are related to POD.

### DESCRIPTIONS OF THE DRAWINGS

The features and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1A is a block diagram of an example configuration within which the systems and methods disclosed herein could be implemented according to some embodiments of the present disclosure;

FIG. 1B is a block diagram illustrating components of an exemplary system according to some embodiments of the present disclosure;

FIG. 2 illustrates an exemplary depiction of the disclosed index according to some embodiments of the present disclosure.

FIG. 3A and FIG. 3B illustrate an exemplary workflow according to some embodiments of the present disclosure;

FIGS. 3C-3O depict non-limiting example embodiments related to disclosed functionality according to some embodiments of the present disclosure;

FIG. 4 illustrates an exemplary embodiment of an overview of a standard;

FIGS. 5A and 5B illustrate various embodiments of communication protocol for communication between one or more controllers and one or more pumps;

FIG. 6 illustrates exemplary interfaces between system components;

FIG. 7 depicts an exemplary implementation of an architecture according to some embodiments of the present disclosure;

FIG. 8 depicts an exemplary implementation of an architecture according to some embodiments of the present disclosure;

FIG. 9 is a block diagram illustrating a computing device showing an example of a client or server device used in various embodiments of the present disclosure;

FIG. 10A depicts an exemplary workflow according to some embodiments of the present disclosure; and

FIG. 10B depicts a non-limiting example embodiment according to some embodiments of the present disclosure.

### DETAILED DESCRIPTION

Electroencephalography (EEG) serves as a crucial tool in monitoring anesthesia by capturing brain activity through recording the electrical signals produced by neurons. During anesthesia administration, EEG monitoring enables anesthesiologists to gauge the depth of anesthesia and the patient's level of consciousness. By analyzing distinct EEG patterns associated with various anesthesia stages, from light sedation to deep anesthesia, anesthesiologists can fine-tune the dosage of anesthetic agents to achieve the desired level of unconsciousness while mitigating the risk of awareness or insufficient anesthesia.

Real-time EEG monitoring facilitates precise adjustments in anesthesia administration, ensuring optimal anesthesia depth tailored to individual patient responses. Moreover, EEG monitoring aids in detecting signs of intraoperative awareness, allowing prompt interventions to deepen anesthesia and prevent patient awareness during surgery. Additionally, monitoring anesthesia-induced changes in brain activity, such as alterations in EEG signal frequency, amplitude, and coherence, enables anesthesiologists to assess the effects of anesthesia on brain function and optimize anesthesia management to minimize adverse effects and complications. In essence, EEG monitoring plays a pivotal role in ensuring the safe and effective delivery of anesthesia, guiding anesthesia management strategies, and enhancing patient outcomes during surgical procedures.

Existing efforts to use EEG monitors, such as the Medtronic Bispectral Index monitor (BIS) monitor to provide EEG-guided anesthetic management has shown mixed results in preventing post-op delirium (POD). Several high quality randomized controlled trials have shown that when anesthesiologists use processed EEG monitoring to reduce anesthetic exposure, the incidence of POD can be reduced by approximately 5 to 9% in absolute terms, an approximately 20 to 36% reduction from a baseline incidence of 25% to 20% or 16%. Other trials showed no improvement in POD from BIS-guided anesthesia management. Overall, across all trials conducted to date, a net reduction averaging 4% from an initial incidence baseline of 25% is observed. This modest outcome can be attributed in part to the limitations of existing anesthetic brain monitors like BIS, which was last updated in 2005, approximately 20 years ago.

As understood by those of ordinary skill in the art, the BIS can be highly inaccurate patients—for example, in older patients who are at highest risk of POD. The BIS was developed using data from young patients and works poorly in older patients, often providing higher values than appropriate, leading anesthesiologists to place patients in burst suppression, a state that is linked to increased risk of post-operative delirium. Furthermore, elderly patients at elevated risk for conditions like dementia or Alzheimer's disease appear to have lower anesthetic requirements than what is predicted by conventional age-adjusted pharmacodynamic models. As such, anesthesiologists, guided by readings from BIS or analogous, conventional monitors, might hesitate to decrease their anesthetic doses even when it might be appropriate. This reluctance could potentially offset the advantages of EEG-guidance, significantly constraining the reduction in anesthetic exposure.

Accordingly, the disclosed systems and methods address such shortcomings, among others, by providing a computerized framework for an improved EEG indices that adapts to specific patient needs, and dynamically adapts to factors of an ongoing procedure to ensure that the proper levels of anesthesia are administered, required and/or maintained. This, as discussed herein in more detail, provides capabilities to prevent, or at least significantly reduce, the onset of POD. Thus, the disclosed systems and methods provide an effective anesthesia management framework that can prevent POD via the DI-based decision support mechanisms discussed herein.

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. As discussed herein, monitoring anesthesia during surgery is crucial to ensure patient safety and optimal surgical outcomes. Anesthesiologists and anesthesia teams employ various techniques and technologies to continuously assess the patient's physiological parameters and adjust anesthesia accordingly. For example, such mechanisms can include, but are not limited to, vital signs monitoring, electrocardiogramalse oximetry, capnography, anesthetic depth monitoring, temperature monitoring, fluid and electrolyte management, and the like.

According to some embodiments, for example, anesthesia monitoring typically begins with the continuous assessment of vital signs, including heart rate, blood pressure, respiratory rate, and oxygen saturation. These parameters provide essential information about the patient's cardiovascular and respiratory status, helping anesthesiologists detect and manage any deviations from normal values promptly. ECG monitoring tracks the electrical activity of the heart, allowing anesthesiologists to identify any abnormalities in cardiac rhythm and intervene if necessary. Continuous ECG monitoring helps ensure cardiovascular stability during surgery.

Pulse oximetry measures the oxygen saturation of arterial blood, providing real-time feedback on the patient's oxygenation status. Monitoring oxygen saturation helps detect hypoxemia (low oxygen levels), which can occur due to factors such as airway obstruction or respiratory depression, and allows prompt intervention to maintain adequate oxygenation.

Capnography measures the concentration of carbon dioxide (CO2) in exhaled breath, providing information about the patient's ventilation and respiratory status. Monitoring end-tidal CO2 levels helps detect hypoventilation or airway obstruction, allowing timely intervention to optimize respiratory function and prevent complications such as hypercapnia (elevated CO2 levels).

In some embodiments, anesthetic depth monitoring can be performed, where various techniques can be used to assess the depth of anesthesia and the patient's level of consciousness during surgery. These may include clinical assessment, EEG monitoring (as discussed in more detail below), or entropy monitoring. Monitoring anesthesia depth helps prevent awareness during surgery while minimizing the risk of excessive anesthesia and its associated complications.

Moreover, in some embodiments, maintaining normothermia (normal body temperature) is essential for patient safety during surgery. Temperature monitoring allows anesthesiologists to detect and prevent perioperative hypothermia or hyperthermia, which can increase the risk of surgical site infections, coagulopathies, and other complications. Additionally, anesthesiologists carefully monitor fluid balance and electrolyte levels during surgery to prevent dehydration, electrolyte imbalances, and hemodynamic instability. Intravenous fluids and electrolyte solutions are administered as needed to maintain adequate hydration and electrolyte balance.

As such, as discussed herein, by continuously monitoring anesthesia and adapting the anesthetic plan based on the patient's physiological responses and surgical requirements, anesthesia teams can optimize patient safety, minimize the risk of complications, and promote successful surgical outcomes. Accordingly, the disclosed systems and methods provide an effective anesthesia management framework that can prevent POD via the disclosed decision support mechanisms, discussed infra. This, among other benefits, can provide healthcare providers with essential tools for comprehensive perioperative care and patient well-being.

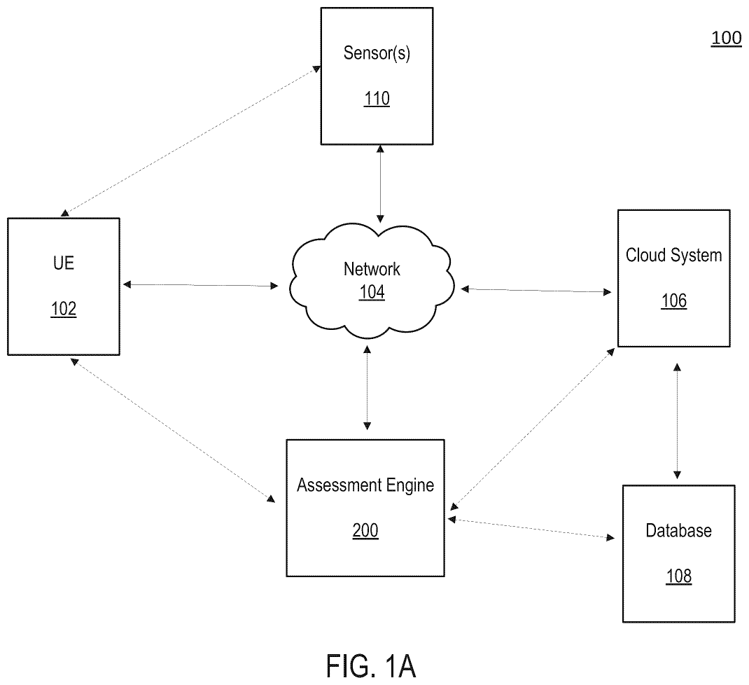

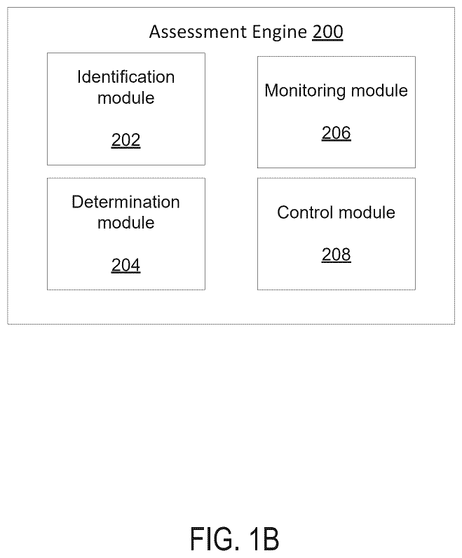

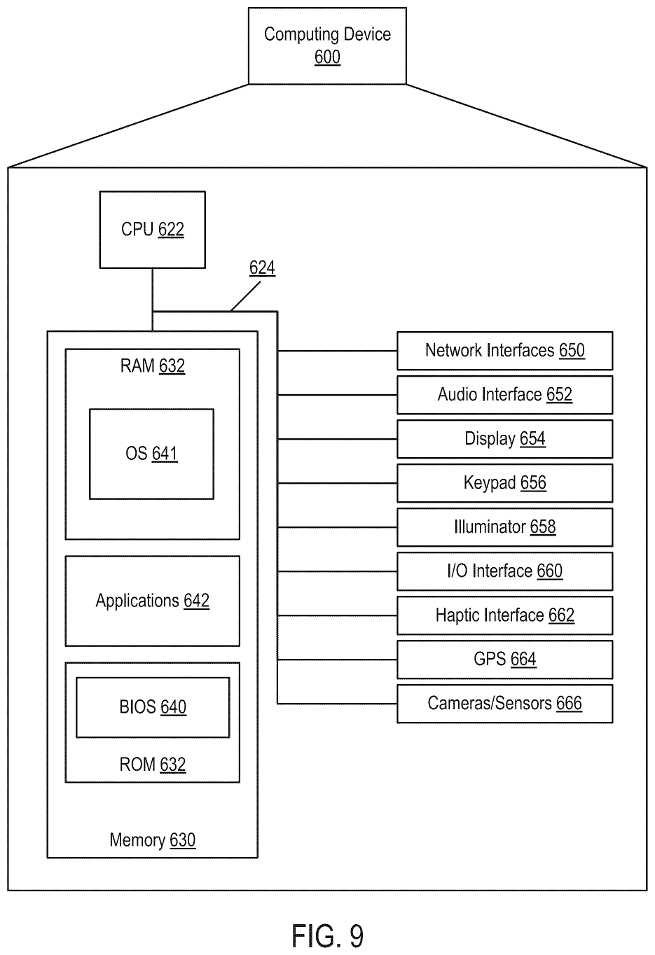

With reference to FIG. 1A, system 100 is depicted which includes user equipment (UE) 102 (e.g., a client device, as mentioned above and discussed below in relation to FIG. 9), network 104, cloud system 106, database 108, sensor(s) 110 and assessment engine 200. It should be understood that while system 100 is depicted as including such components, it should not be construed as limiting, as one of ordinary skill in the art would readily understand that varying numbers of UEs, cloud systems, databases, computer systems and/or networks can be utilized; however, for purposes of explanation, system 100 is discussed in relation to the example depiction in FIG. 1A.

According to some embodiments, UE 102 can be any type of device, such as, but not limited to, a wearable device, mobile phone, tablet, laptop, Internet of Things (IoT) device, surgical robot, autonomous machine, and any other type of modern device, and/or any other device equipped with a cellular or wireless or wired transceiver.

In some embodiments, UE 102 can have associated therewith a plurality of sensors 110 to collect data from a patient. In some embodiments, sensors 110, for example, can be any type of known or to be know sensor device that enables monitoring a patient's vitals, such as, but not limited to, pulse oximetry, electrocardiogram, monitoring of non-invasive blood pressure, end-tidal carbon dioxide, airway pressure and the like.

According to some embodiments, as discussed herein, sensors 110 correspond to measurement tools for monitoring a characteristic, feature, attribute, value and/or metric associated with a UE 102 or patient. According to some embodiments, one or more of the sensors 110 may include, but are not limited to, an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, and the like, and/or any combination thereof. Further discussion and examples of such sensors are discussed below.

According to some embodiments, sensors 110 may be integrated into the operation of the UE 102 in order to monitor the status of a patient. In some embodiments, the data acquired by the sensors 104 may be used to train a machine learning and/or artificial intelligence (ML/AI) algorithm used by the UE 102 and/or artificial intelligence to control the UE 102, as discussed below. According to some embodiments, such ML/AI can include, but are not limited to, computer vision, neural network analysis, and the like, as discussed below.

In some embodiments, network 104 can be any type of network, such as, but not limited to, a wireless network, cellular network, the Internet, and the like (as discussed above). Network 104 facilitates connectivity of the components of system 100, as illustrated in FIG. 1A.

According to some embodiments, cloud system 106 may be any type of cloud operating platform and/or network based system upon which applications, operations, and/or other forms of network resources may be located. According to some embodiments, cloud system 106 can be any type of secure local and/or network device, location, application, account, portal, resource, and the like, upon which patient assessment and/or medical monitoring is being performed. For example, system 106 can involve and/or be associated with, a medical or service provider (or third party provider), and have an associated, but not limited to, a web-portal, website, application, account, datastore, repository, cloud, peer device, platform, exchange, and the like, or some combination thereof. For example, system 106 can represent the cloud-based architecture associated with a medical provider, which has associated network resources hosted on the internet or private network (e.g., network 104), which enables (via engine 200) the patient management discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, and the services and applications provided by cloud system 106 and/or assessment engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

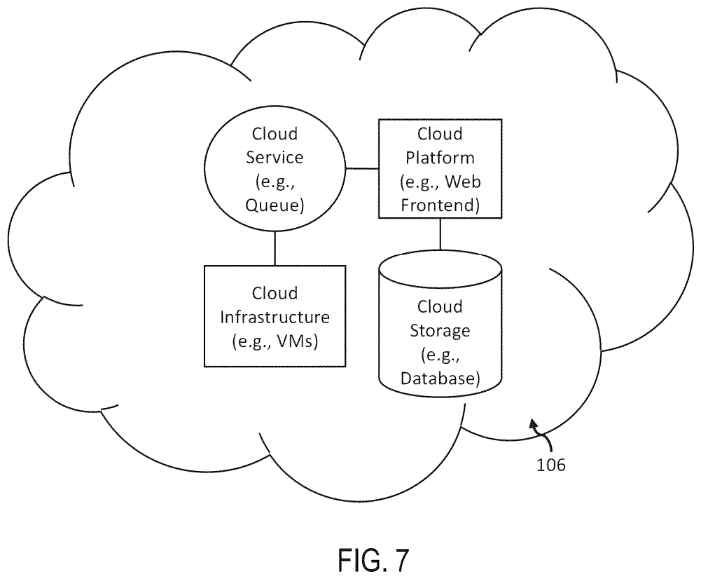

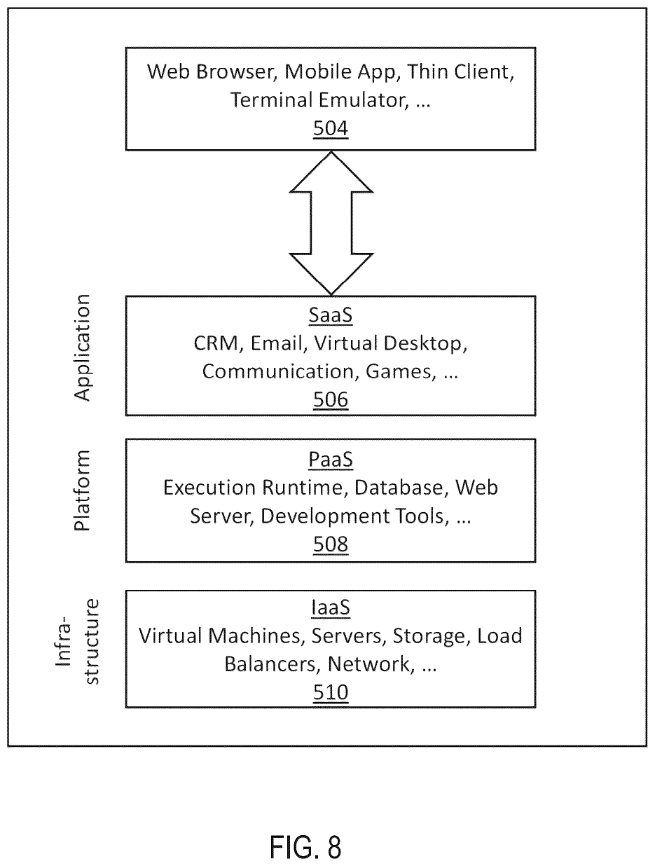

Turning to FIG. 7 and FIG. 8, in some embodiments, the exemplary computer-based systems/platforms, the exemplary computer-based devices, and/or the exemplary computer-based components of the present disclosure may be specifically configured to operate in a cloud computing/architecture 106 such as, but not limiting to: infrastructure as a service (IaaS) 510, platform as a service (PaaS) 508, and/or software as a service (SaaS) 506 using a web browser, mobile app, thin client, terminal emulator or other endpoint 504. FIG. 7 and FIG. 8 illustrate schematics of non-limiting implementations of the cloud computing/architecture(s) in which the exemplary computer-based systems for administrative customizations and control of network-hosted application program interfaces (APIs) of the present disclosure may be specifically configured to operate.

Turning back to FIG. 1A, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository

Assessment engine 200, as discussed above and further below in more detail, can include components for the disclosed functionality. According to some embodiments, assessment engine 200 may be a special purpose machine or processor, and can be hosted by a device on network 104, within cloud system 106 and/or on UE 102. In some embodiments, engine 200 may be hosted by a server and/or set of servers associated with cloud system 106.

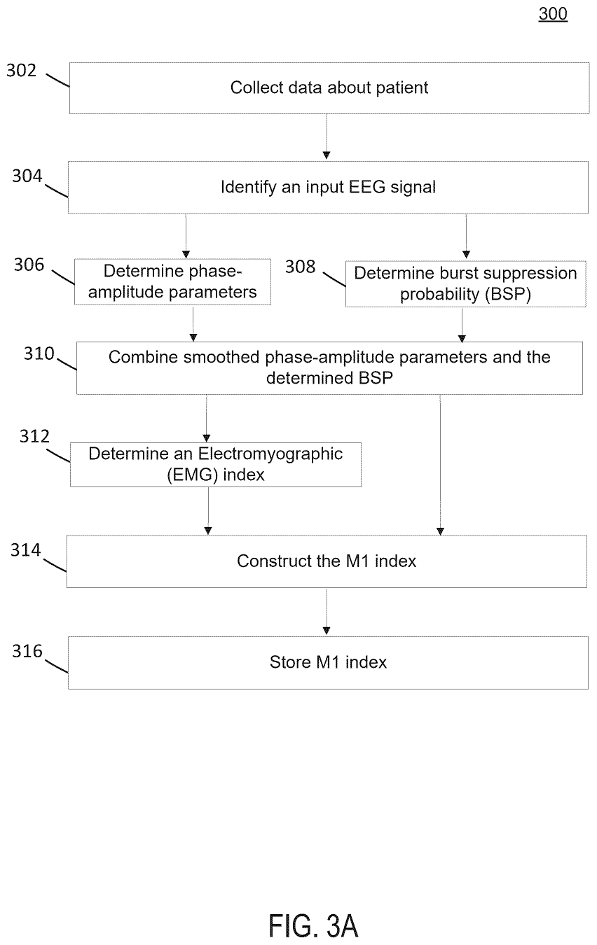

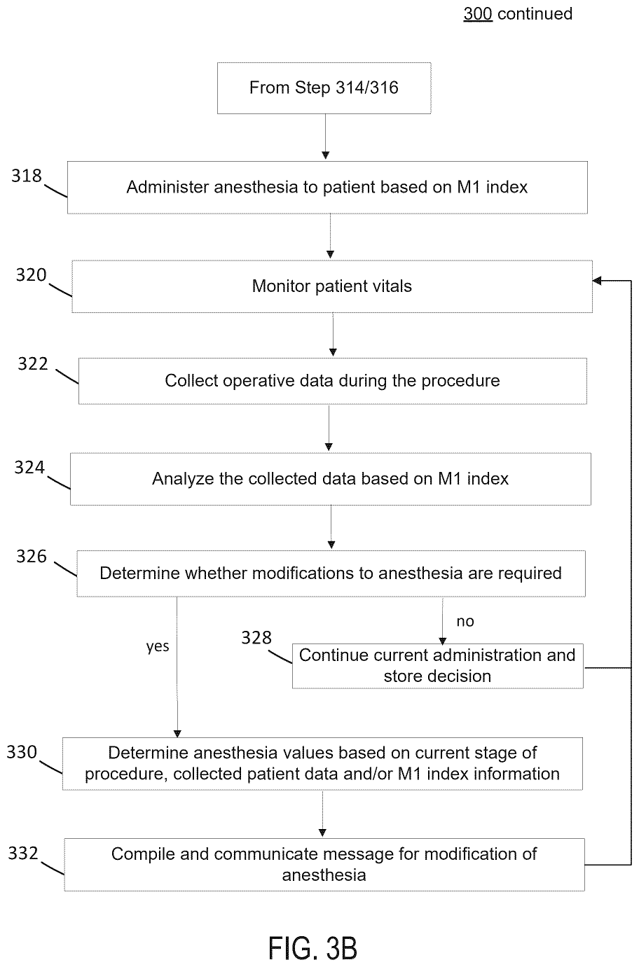

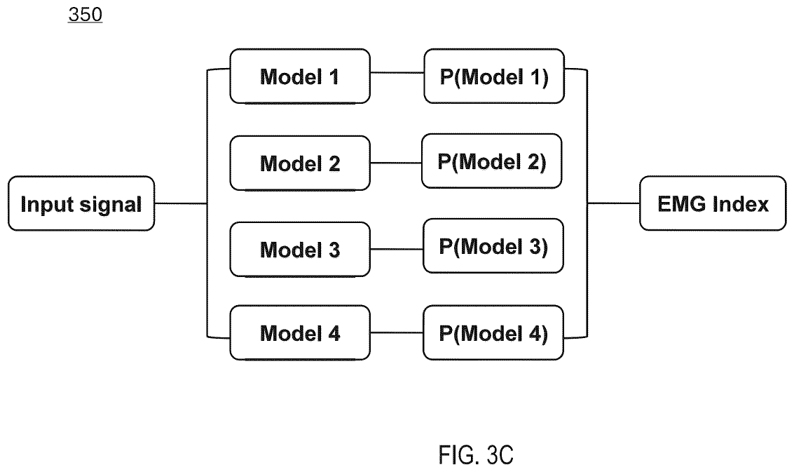

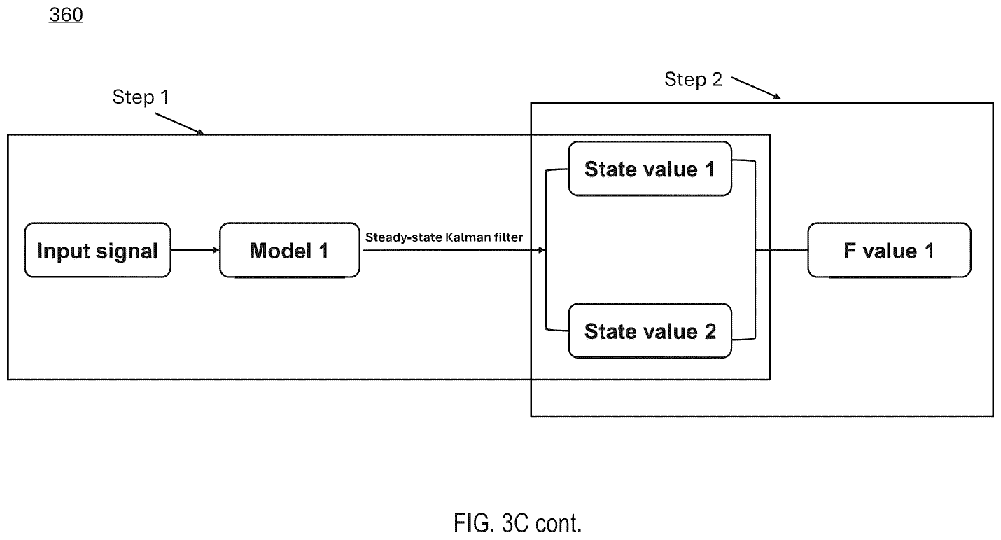

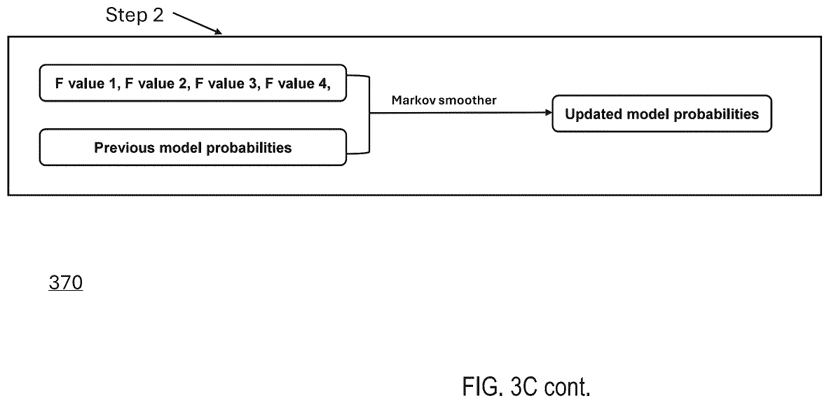

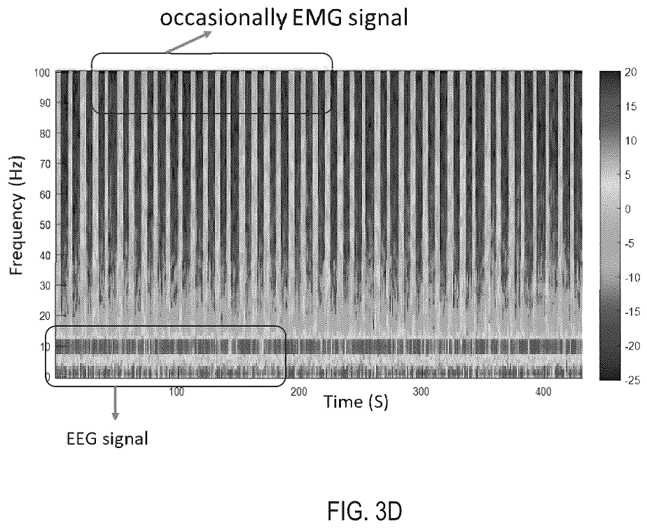

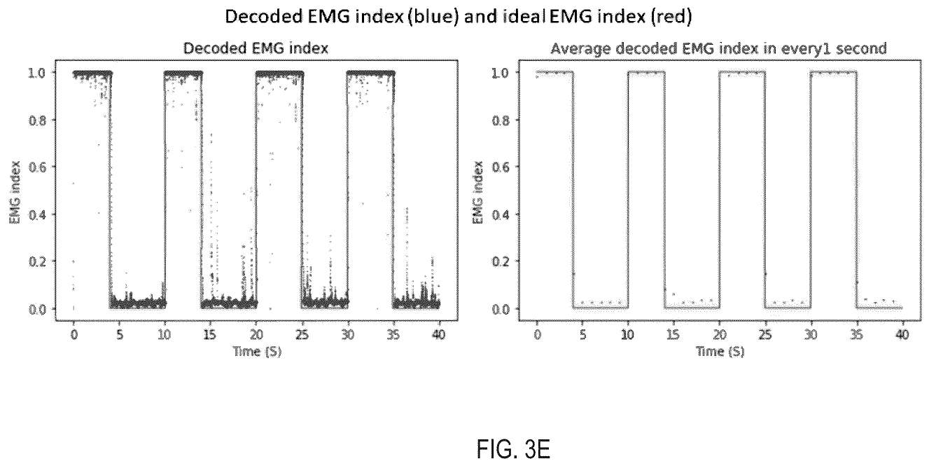

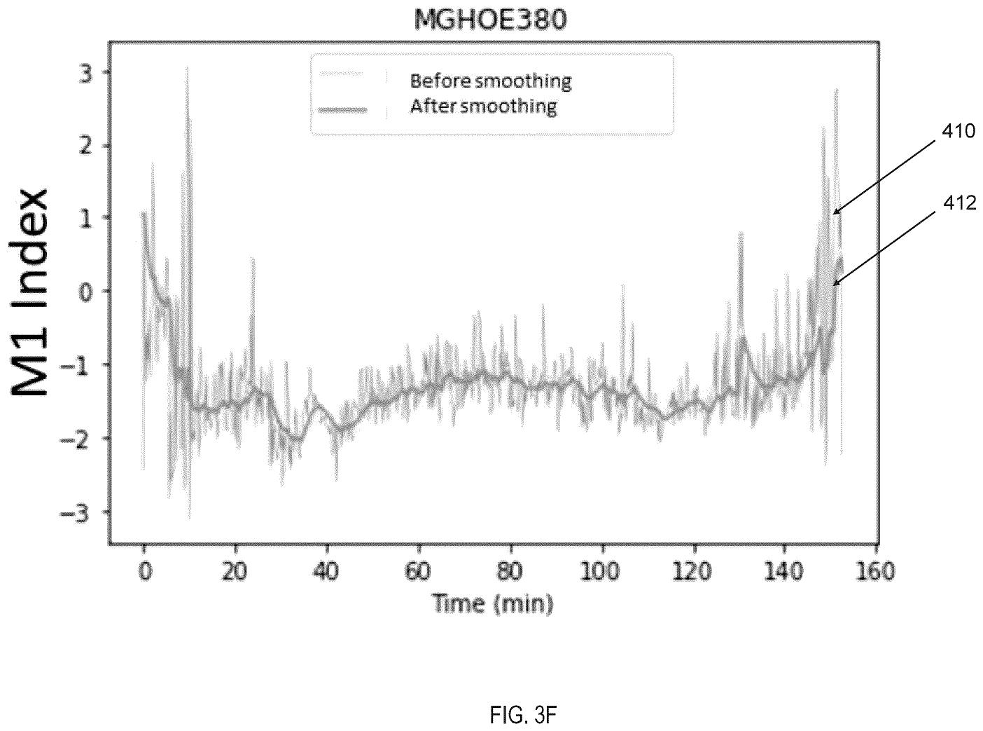

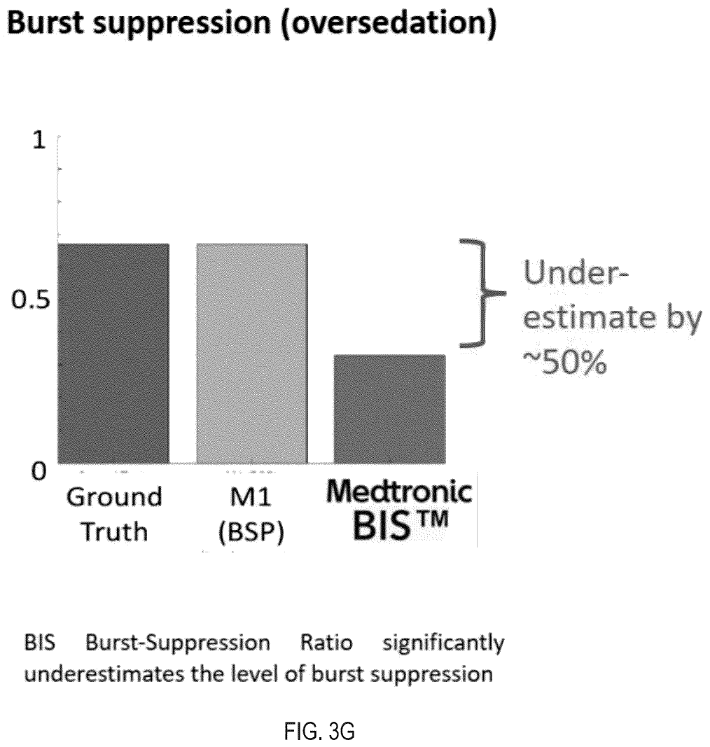

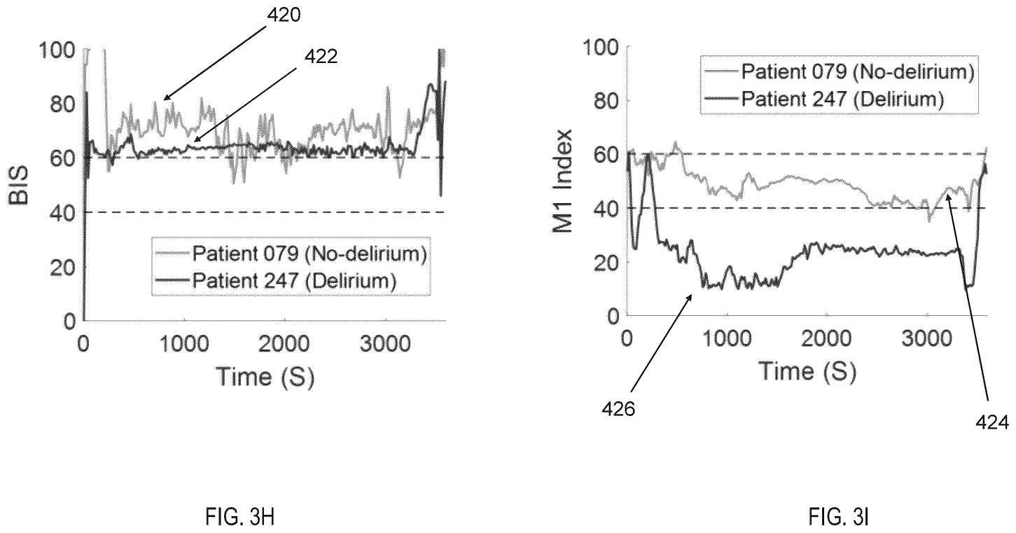

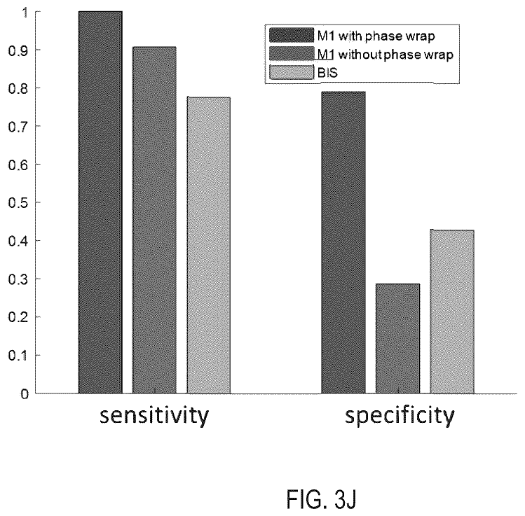

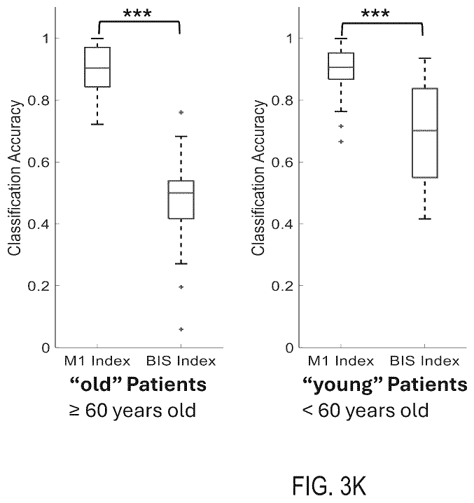

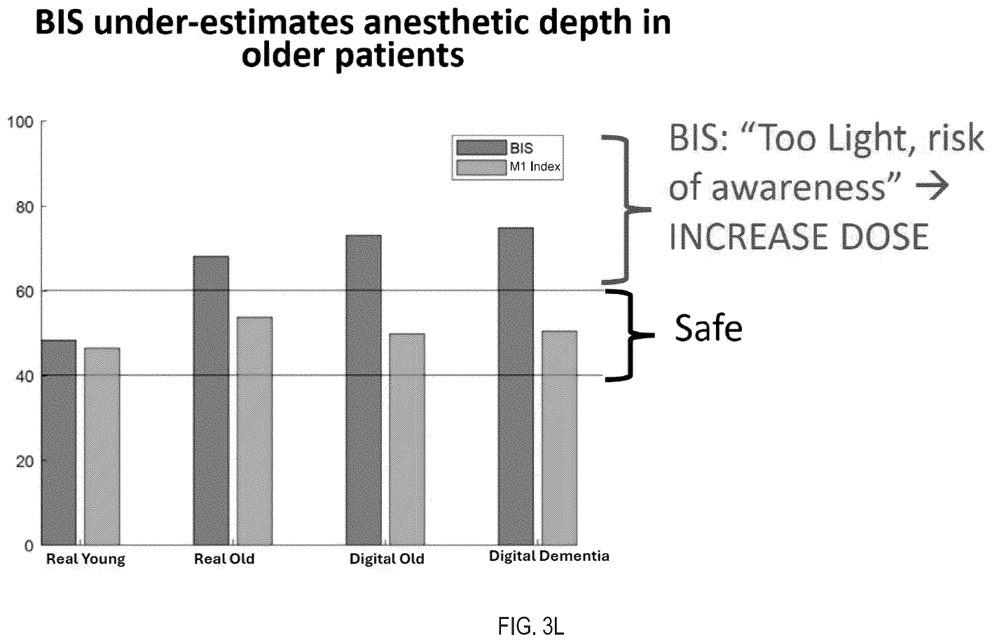

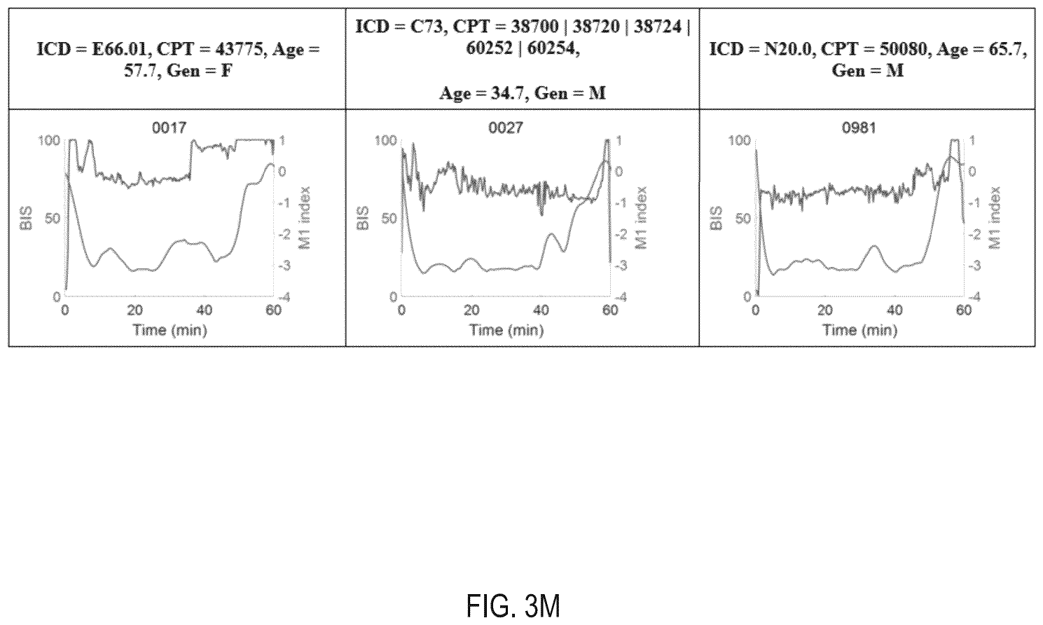

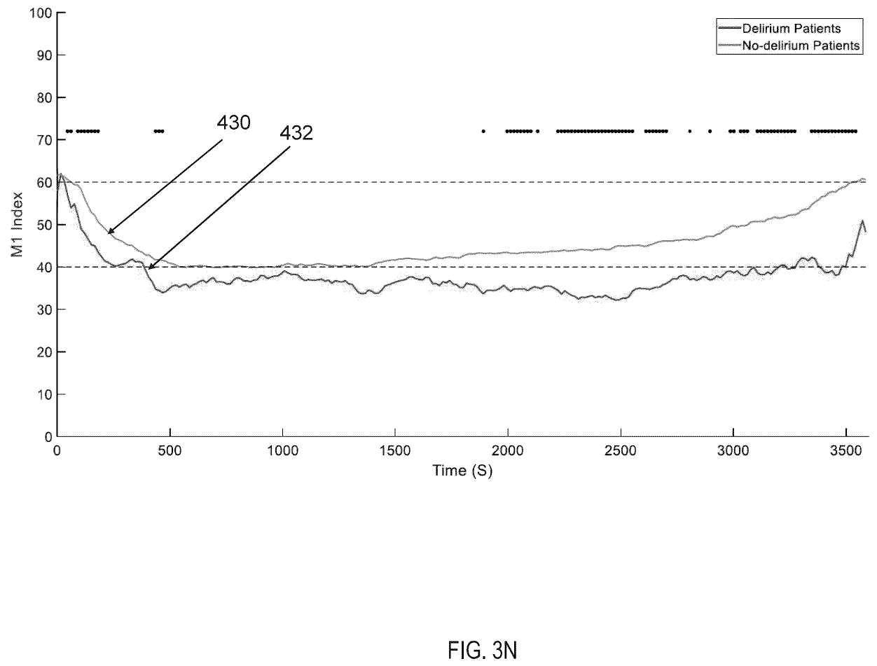

According to some embodiments, as discussed in more detail below, assessment engine 200 may be configured to implement and/or control a plurality of services and/or microservices, where each of the plurality of services/microservices are configured to execute a plurality of workflows associated with performing the disclosed media management. Non-limiting embodiments of such workflows are provided below in relation to at least FIGS. 3A and 3B.

According to some embodiments, as discussed above, assessment engine 200 may function as an application provided by cloud system 106. In some embodiments, engine 200 may function as an application installed on a server(s), network location and/or other type of network resource associated with system 106. In some embodiments, engine 200 may function as an application installed and/or executing on UE 102. In some embodiments, such application may be a web-based application accessed by UE 102 and/or other devices over network 104 from cloud system 106. In some embodiments, engine 200 may be configured and/or installed as an augmenting script, program or application (e.g., a plug-in or extension) to another application or program provided by cloud system 106 and/or executing on UE 102.

As illustrated in FIG. 1B, according to some embodiments, assessment engine 200 includes identification module 202, determination module 204, monitoring module 206 and control module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

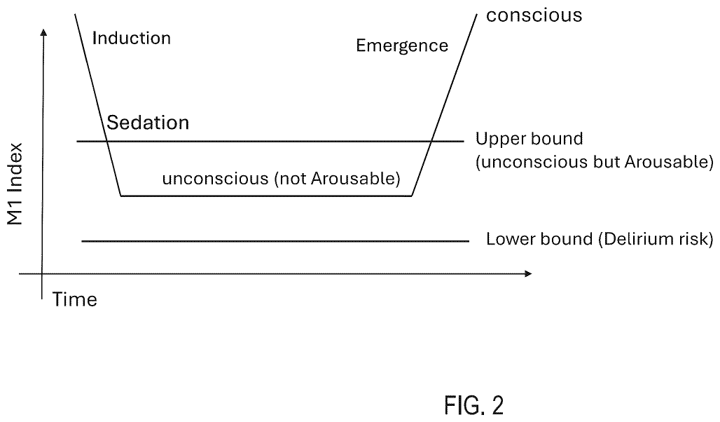

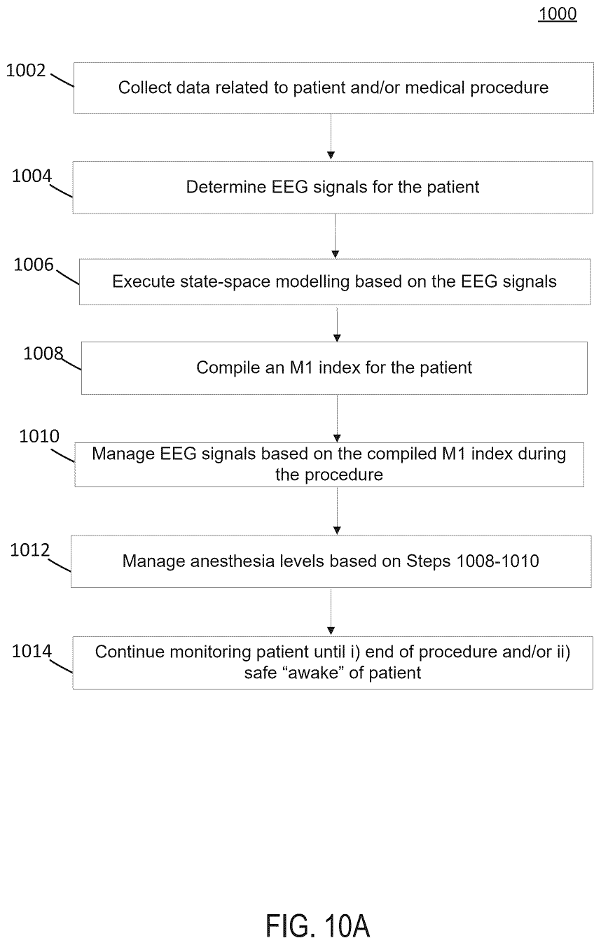

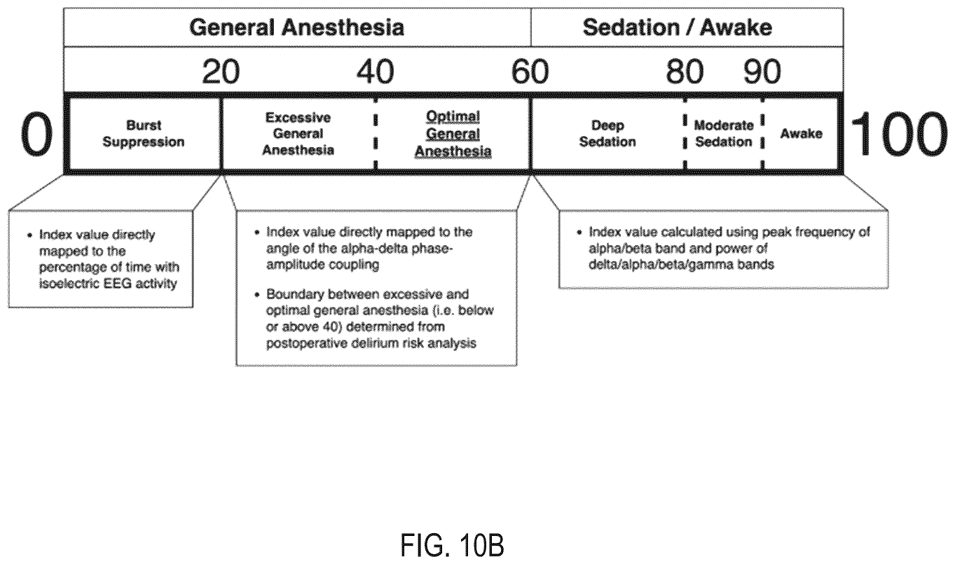

FIG. 2 provides an illustrative depiction of how the disclosed framework can provide improved POD prevention. As provided in more detail below with reference to FIG. 3A, FIG. 3B, FIG. 10A and FIG. 10B, an enhanced brain state index (referred to as the “M1 index”) can be compiled and leveraged for perioperative tasks to detect the following states: sedation, unconscious (not Arousable), unconscious but arousable, and conscious (which can have an upper bound and lower bound), which are related to under sedation (wake up risk) and over sedation (delirium risk). An example of such is depicted in FIG. 2.

Turning to FIGS. 3A and 3B, Process 300 provides non-limiting example embodiments for the disclosed patient management framework. According to some embodiments, Steps 302, 304 and 318 can be performed by identification module 202 of assessment engine 200; Steps 306-312 and 324-330 can be performed by determination module 204; Steps 314, 316 and 332 can be performed by control module 208; and Steps 320 and 322 can be performed by monitoring module 206.

It should be understood that while the discussion herein may focus on prevention and/or mitigation of POD via the M1 index, inter alia, it should not be construed as limiting, as one of ordinary skill in the art would understand that the determination and real-time application of the M1 index, as discussed with reference to the steps of Process 300, discussed infra, can apply to any type of known or to be know side effect of anesthesia or other type of medicine, whether a medical condition and/or mental health issue, such as, but not limited to, consciousness, POD, depression, POCD, PONV, respiratory complications, cardiovascular issues, chronic postoperative pain syndromes, thromboembolism, endocrine and metabolic disturbances, allergic reactions or adverse drug reactions, postoperative fever, postoperative ileus, peripheral nerve injuries, and the like.

According to some embodiments, Process 300 begins with Step 302 where engine 200 can collect data about a patient (also referred to as a user, used interchangeably). According to some embodiments, such data can correspond to comprehensive information about the patient's medical history, current health status, and any relevant preoperative assessments. For example, this can include, but is not limited to, age, weight, height, and baseline physiological parameters. In some embodiments, such data may also refer to, but is not limited to, any preexisting medical conditions, such as cardiovascular disease, respiratory disorders, neurological conditions, allergies, and endocrine disorders, as well as any previous surgeries or anesthesia-related complications. Even further, the data can provide information related to the patient's medical history, which can include, for example, prescription medications, over-the-counter drugs, herbal supplements, and recreational substances, and the like.

In some embodiments, the data may be related to information for identifying potential drug interactions or contraindications. For example, laboratory test results, such as blood tests, ECG, imaging studies, and preoperative evaluations, can provide valuable insights into the patient's overall health status and help assess surgical risk. In some embodiments, the data may also provide information related to, but not limited to, the patient's airway anatomy, dentition, and potential risk factors for difficult intubation or airway management.

As per Step 302, understanding the patient's baseline functional status, cognitive function, and/or psychological well-being can be essential for tailoring anesthesia management to individual needs and ensuring optimal perioperative care. In some embodiments, such collected data can be stored in database 108, as discussed above.

In Step 304, an EEG signal can be identified and analyzed. As discussed above, EEG is a vital tool used during anesthesia to monitor the brain's electrical activity and assess the depth of anesthesia and the patient's level of consciousness. By analyzing the EEG patterns generated by neurons in the brain, anesthesiologists can gauge the patient's response to anesthetic agents and make real-time adjustments to maintain the desired level of unconsciousness while minimizing the risk of awareness.

Different stages of anesthesia can be associated with characteristic EEG patterns, ranging from low-frequency, high-amplitude waves during deep anesthesia to higher-frequency, lower-amplitude waves during lighter stages of sedation. As discussed herein, continuous EEG monitoring allows anesthesiologists to track changes in brain activity throughout surgery and adapt anesthesia administration accordingly. Additionally, EEG monitoring helps detect signs of intraoperative awareness, enabling prompt interventions to deepen anesthesia and prevent patient discomfort or distress. By providing objective data on the patient's neurophysiological state, EEG enhances the safety and efficacy of anesthesia management, ensuring optimal patient outcomes and minimizing the risk of complications.

Accordingly, as in Step 304, EEG signal inputs can be identified, which are recordings of the brain's electrical activity, captured non-invasively through electrodes placed on the scalp. These signals convey crucial information about the brain's functioning, characterized by their frequency, amplitude, and waveform patterns.

For example, different frequency bands within EEG signals correspond to specific states of brain activity: slow (0.1 to 1 Hz) and delta waves (0.5-4 Hz) are associated with deep sleep and unconsciousness, theta waves (4-8 Hz) prevail during drowsiness and light sleep, alpha waves (8-12 Hz) are prominent during wakefulness and relaxation with closed eyes, beta waves (12-30 Hz) signify alertness and active cognitive processing, and gamma waves (>30 Hz) are linked to complex cognitive functions like perception and memory. In some cases, due to their combined presence during sleep and unconsciousness, the presence of either slow and delta waves may be referred to as a slow/delta wave (0.1 to 4 Hz).

Amplitude reflects the intensity of neuronal activity, with higher amplitudes indicating synchronized activity and lower amplitudes suggesting background or desynchronized activity.

Additionally, EEG signals exhibit characteristic waveform patterns, including sharp waves, spikes, and rhythmic oscillations, which provide insights into brain states, cognitive processes, and pathological conditions, such as, for example, epilepsy. Thus, the EEG signals provide valuable insights into the brain's electrical activity, which can provide an assessment as to a brain's function.

Thus, the EEG signals collected in Step 304 can be stored in database 108, as discussed above, and can be for a predetermined period of time and/or for a type of event (e.g., a detected type of brain activity), and the like, or some combination thereof.

In some embodiments, upon performing Step 304, engine 200 can execute Step 304 and Step 306, which can be performed iteratively (in either order), simultaneously and/or substantially simultaneously (e.g., with an overlap in their processing).

In Step 306, engine 200 can calculate phase-amplitude parameters (k_{mod}, phi_{mod} and phiₘᵢₙ) from the EEG signal (from Step 304). K_{mod} is a parameter that defines how strong the alpha amplitude changes based on the phase of the slow/delta wave. Phi_{mod} is a parameter that defines when the alpha amplitude is maximum, where the phase of the slow/delta wave is. Phiₘᵢₙ is a parameter that defines when the alpha amplitude is maximum, where the phase of the slow/delta wave is.

In some embodiments, engine 200 can perform the phase-amplitude parameter computation by deploying a bifurcated switching state space model, diverging from the conventional singular model used in current systems. In some embodiments, two distinctive sub-models can be incorporated, which can respectively address the upper and lower boundaries for slow and alpha oscillations. According to some embodiments, such structure enhances the robustness of phase-amplitude parameter estimation across diverse patient alpha and slow frequencies. An example of such computational processing performed by engine 200 is provided below:

$$Extract {x}_{st} , {x}_{ft} from a switching - state - space model with 2 sub models$$

$$The 2 sub - models have the same observation equation :$$

$${y}_{t} = {x}_{s 1 t} + {x}_{f 1 t} + {r}_{t} , {r}_{t} ∼ N ( 0 , R )$$

$$The 2 sub - models have different state models :$$

$${x}_{t} = {A}_{eeg} {x}_{t - 1} + {q}_{t} , {q}_{t} ∼ ( 0 , Q )$$

$$Model 1 has f\_slow1 = 0.4 Hz , and f\_fast1 = 7 Hz .$$

$$Model 2 has f\_slow2 = 2.1 Hz and f\_fast2 = 15 Hz .$$

$${x}_{t} = {A}_{eeg} {x}_{t - 1} + {q}_{t} , {q}_{t} ∼ N ( 0 , Q )$$

$${{A}_{eeg}}^{} = a [ \begin{matrix}{A}_{fast} & \begin{matrix}0 & 0 \\ 0 & 0\end{matrix} \\ \begin{matrix}0 & 0 \\ 0 & 0\end{matrix} & {A}_{slow}\end{matrix} ]$$

$${A}_{fast} = [ \begin{matrix}cos ( 2 π {f}_{fast} / {f}_{sample} ) & - sin ( 2 π {f}_{fast} / {f}_{sample} ) \\ sin ( 2 π {f}_{fast} / {f}_{sample} ) & cos ( 2 π {f}_{fast} / {f}_{sample} )\end{matrix} ]$$

$${A}_{slow} = [ \begin{matrix}cos ( 2 π {f}_{fast} / {f}_{sample} ) & - sin ( 2 π {f}_{slow} / {f}_{sample} ) \\ sin ( 2 π {f}_{slow} / {f}_{sample} ) & cos ( 2 π {f}_{slow} / {f}_{sample} )\end{matrix} ]$$

Accordingly, the information determined in Step 306 can be stored in database 108, as discussed above.

In Step 308, engine 200 can determine a burst suppression probability (BSP), which can be stored in database 108. According to some embodiments, BSP is a metric used in EEG monitoring to quantify the depth of anesthesia or sedation. BSP represents the likelihood that a patient's EEG signal exhibits a burst suppression pattern, characterized by alternating periods of high-amplitude activity (bursts) followed by periods of very low or absent activity (suppression). Such pattern can be typically observed when the brain's activity is profoundly suppressed, such as during deep anesthesia or coma.

According to some embodiments, BSP can be calculated based on the percentage of time that the EEG signal spends in the suppression state over a defined period, often measured in seconds or minutes. In some embodiments, a higher BSP value indicates a greater proportion of time spent in burst suppression, reflecting deeper levels of anesthesia or sedation.

As discussed herein, BSP can be utilized as a quantitative measure to assess the adequacy of anesthesia or sedation, particularly during procedures requiring deep levels of unconsciousness, such as major surgery or neurocritical care. By monitoring BSP alongside other EEG parameters, the dosage of anesthetic agents can be dynamically triaged to achieve the desired depth of anesthesia while minimizing the risk of awareness, inadequate sedation and/or POD, inter alia.

Accordingly, the BSP determined in Step 308 provides a numerical assessment (e.g., a parameter) of the extent to which a patient's brain activity is suppressed during anesthesia or sedation. According to some embodiments, BSP can be a parameter from 0 to 1, which defines how strong the burst suppression is. In some embodiments, there is no suppression signal when BSP=0; and there is a suppression signal when BSP=1.

In Step 310, engine 200 can combine the smoothed phase-amplitude parameters (the phase-amplitude parameters from Step 306, which are then smoothed, as discussed herein) with the determined BSP (from Step 308).

According to some embodiments, engine 200 amalgamates the BSP with the phase-amplitude parameter phiₘᵢₙ. According to some embodiments, the inclusion of the BSP can be two-fold: i) a high BSP implies that the alpha energy is predominantly low (e.g., at or below a threshold), which owing to a reduced signal-to-noise ratio (SNR), renders the phase-amplitude parameters as inaccurate, thereby necessitating an alternative feature to accurately estimate the brain state of the patient; and ii) a high burst suppression robustly indicates an oversedated state of the patient, enabling precise estimation of the brain state at that moment.

An example of the performance of the BSP algorithm is depicted in in FIG. 3G. As shown in FIG. 3G, burst suppression detection using the BSP algorithm provides far a more accurate characterization of burst suppression compared with the BIS algorithm, estimating the true underlying proportion of suppression periods from simulated burst suppression data. Meanwhile, the BIS algorithm underestimates the amount of suppression by approximately 50%.

According to some embodiments, as mentioned above, the phase-amplitude parameters can be smoothed. In some embodiments, such smoothing can involve utilizing a filter with an integrated phase wrap to smooth the phi_{mod}. This filter could take the form of a Kalman filter (e.g., a random walk Kalman filter where the hidden state of interest is modeled as a random walk, or a steady-state Kalman filter) or other linear or non-linear filter, for example. An example of such smoothing is depicted in FIG. 3F, which illustrates how the smoothing reduces the moment to moment noisy variation in the M1 index (for example, lines 410, 412 before and after smoothing, respectively). This smoothing or phase wrap and the subsequent reduction in variation improves the sensitivity and specificity of M1 for predicting POD as shown in FIG. 3J. Also depicted in FIG. 3J, M1 in either form, with or without smoothing or phase wrap, outperforms BIS. In some embodiments, during the computation of the observation difference for a random walk Kalman filter, the phase wrap ensures the observation difference adheres to a range of negative pᵢ to pᵢ, which can be effectuate via the following methodology:

The observation difference at time t is defined as yₜ−xₜ₋₁ where yₜ is the observation (M1 without phase wrap) at time t and xₜ₋₁ is the state (M1 after phase wrap) at time t−1. If yₜ−xₜ₋₁<−π, observation difference=yₜ−xₜ₋₁+2π. If yₜ−xₜ₋₁>π, observation difference=yₜ−xₜ₋₁−2π.

Thus, according to some embodiments, engine 200 can maintain the observation difference as-is if negative pᵢ is less than the observation difference and the observation difference is less than pᵢ;

If the observation difference is less than negative pᵢ, adjust by: observation difference equals observation difference plus 2 times pᵢ

If the observation difference is greater than pi, adjust by: observation difference equals observation difference minus 2 times pᵢ.

Accordingly, in some embodiments, such methodology affords a considerably smoother and more interpretable index than BIS, as discussed herein (e.g., the M1 index discussed infra).

According to some embodiments, the construction of the M1 index (as in Step 314) can be performed based on the combination performed in Step 308. Thus, according to some embodiments, the synthesis of the M1 index can be achieved as follows, via Step 314 (from Step 310, as depicted in FIG. 3A):

$$Combined index = min ( {phi}_{min} , {index}_{bsp} ) , \qquad Eq . 1$$

where, index_{bsp}=pᵢ, when burst suppression probability (BSP)<0.1
index_{bsp}=−pᵢ*sqrt {(BSP-0.1)/(1−0.1)}, when BSP >=0.1.

In some embodiments, the M1 index can be constructed based further on a determined electromyographic (EMG) index for the patient, as in Step 312. An example of the burst suppression discussed herein is depicted in in FIG. 3G.

In Step 312, engine 200 can execute a switching state-space model, which can determine and eliminate muscle artifacts within the EEG signal (from Step 304) through the establishment of an EMG Index, which can be scaled from 0 to 1. Examples of the detection of muscle artifacts in the EEG signal(s) is depicted in FIG. 3D and FIG. 3E. In some embodiments, a score of 0 indicates a 0% probability of concurrent EMG signal presence, while a score of 1 signifies a 100% probability. According to some embodiments, validation of the EMG Index can be executed via a set of simulations utilizing signals composed of EEG with intermittent EMG interference. Thus, the EMG Index can accurately identify muscle artifacts in EEG signals.

According to some embodiments, with reference to FIG. 3C, a switching state-space model can be used to calculate the EMG index. As depicted in example 350 of FIG. 3C, a state-space model with 4 sub-models can be designed and assumed that, at every time step, the input signal is a linear combination of 4 models. It should be understood that n models/sub-models can be utilized without departing from the scope of the instant disclosure. In some embodiments, the model probability, referred to as P(model i), can be calculated at each step. For example:

$$P ( model 1 ) + P ( model 2 ) + P ( model 3 ) + P ( model 4 ) = 1. \qquad Eq . 2$$

$$Where , the EMG index = ( 1 - P ( model 1 ) ) * 100. \qquad Eq . 3$$

Further, as depicted in examples 360 and 370 in FIG. 3, the EMG index determination can involve three (3) steps (Step 1, Step 2, Step 3). First, in Step 1 (inclusive of the components captured in the “box” of Step 1), using the input signal, engine 200 can calculate the state values and state covariance for every model using a steady-state Kalman filter. Next, in Step 2, engine 200 can calculate the F value for each model using state value and state covariance. Then, Step 3, engine 200 can update a model probability using Markov smoother based on the previous model probabilities and the F values.

Accordingly, in some embodiments, the model probability at time 0 can be represented as:

$$P ( mode 1 ) = 1 , P ( mode 2 ) = P ( mode 3 ) = P ( mode 4 ) = 0. \qquad Eq . 4$$

According to some embodiments, utilizing example n models as 4, 4 models and sub-models can be designed via the same form:

$$Observation model : {y}_{t}^{j} = {H}_{j} {x}_{t}^{j} + {r}_{jt} , {r}_{jt} \sim N ( 0 , {R}_{j} ) \qquad Eq . 5$$

$$State model : {x}_{t}^{j} = {A}_{j} {x}_{t - 1}^{j} + {q}_{jt} , {q}_{jt} \sim N ( 0 , {Q}_{j} ) ,$$

Where j is the model index, j=1, 2, 3, or 4;
yⱼₜ is a scalar, which represents the observation signal at time step t. xⱼₜ is the state variable;
Hⱼ, Aⱼ, Qⱼ, and Rⱼ are model parameters;
Aⱼ is the transition matrix; Hⱼ is the observation matrix; Qⱼ is the transition covariance; and Rⱼ is the observation covariance.

According to some embodiments, each model can share the same observation covariance,
Rⱼ=1 for j=1,2,3 and 4.

IN some embodiments, the first model can be an EEG-only model:
A₁ is a 4*4 matrix. A₁=A_{eeg}; H₁ is a 4*1 vector; xₗₜ is a 4*1 vector; and Q₁ is a 4*4 identity matrix.

In some embodiments, models 2, 3 and 4 can be the EEG+EMG model. In some embodiments, they can share the same transition matrices and observation matrices. That is:
A₂=A₃=A₄, and H₂=H₃=H₄. x₂ₜ, x₃ₜ and x₄ₜ are 6*1 vector. For example:

$${H}_{2} = {H}_{3} = {H}_{4} = {[ 1 , 0 , 1 , 0 , 1 , 0 ]}^{T} . \qquad Eq . 6$$

In some embodiments, A₂ can be a 6*6 matrix:

$${A}_{2} = [ \begin{matrix}{A}_{eeg} & 0 \\ 0 & {A}_{emg}\end{matrix} ] . \qquad Eq . 7$$

In some embodiments, Q₂ can be a 6*6 matrix:

$${Q}_{2} = [ \begin{matrix}1 & 0 & 0 & 0 & 0 & 0 \\ 0 & 1 & 0 & 0 & 0 & 0 \\ 0 & 0 & 1 & 0 & 0 & 0 \\ 0 & 0 & 0 & 1 & 0 & 0 \\ 0 & 0 & 0 & 0 & {E}_{2} & 0 \\ 0 & 0 & 0 & 0 & 0 & 0\end{matrix} ] . \qquad Eq . 8$$

In some embodiments, E₂=emg_{engery_m2}, which can control the EMG energy level of model 2, model 3 and model 4 using the same parameters as in model 2 except their transition_{covariance} can correspond to Q₃ and Q₄, as depicted below:

$${Q}_{3} = [ \begin{matrix}1 & 0 & 0 & 0 & 0 & 0 \\ 0 & 1 & 0 & 0 & 0 & 0 \\ 0 & 0 & 1 & 0 & 0 & 0 \\ 0 & 0 & 0 & 1 & 0 & 0 \\ 0 & 0 & 0 & 0 & {E}_{3} & 0 \\ 0 & 0 & 0 & 0 & 0 & 0\end{matrix} ] {Q}_{4} = [ \begin{matrix}1 & 0 & 0 & 0 & 0 & 0 \\ 0 & 1 & 0 & 0 & 0 & 0 \\ 0 & 0 & 1 & 0 & 0 & 0 \\ 0 & 0 & 0 & 1 & 0 & 0 \\ 0 & 0 & 0 & 0 & {E}_{4} & 0 \\ 0 & 0 & 0 & 0 & 0 & 0\end{matrix} ] . \qquad Eq . 9$$

In some embodiments, the EMG energy levels of models 2, 3 and 4 can be different. For example, as above, provided are how models 1, 2, 3, and 4 are defined. In some embodiments, after having all 4 models with known parameters, the real-time processing example is:

$$decode\_state \_ms , emg\_index \_ave , \\ emg\_index = eeg\_and \_emg \_markov . steady\_full \_data \backslash \\ ( observation\_segment , bin\_size \_poi \vert nts , markov\_matrix , m 1 , m 2 , m 3 , m 4 )$$

In some embodiments, the inputs can be as follows:
observation_segment: the observation signal;
bin_size_points: How many points are used to calculate the average EMG index;
markov_matrix: the matrix used for Markov smoother, where the Markov matrix M for EMG index calculation is:

$$M = [ \begin{matrix}0.999 & 0.001 / 3 & 0.001 / 3 & 0.001 / 3 \\ 0.001 & 0.999 / 3 & 0.999 / 3 & 0.999 / 3 \\ 0.001 & 0.999 / 3 & 0.999 / 3 & 0.999 / 3 \\ 0.001 & 0.999 / 3 & 0.999 / 3 & 0.999 / 3\end{matrix} ] . \qquad Eq . 10$$

In some embodiments, the EMG index can be defined as the probability for all EMG models, which is 1−the probability for EEG only model. According to some embodiments, the inputs can be: m₁,m₂,m₃,m₄: 4 models explained in model construction part, discussed supra.

In some embodiments, the outputs are:
decode_{state_ms}: decoded state using switching state-space model;
emg_{index_ave}: average EMG index using every bin_{size_points} of data; and
emg_{index}: EMG index at every sample point.

According to some embodiments, at each time step, first, the 4 steady-state Kalman filters with the same observation are executed, which results in determining the state estimation xⱼₜ and the state covariance Pⱼₜ for each model (Step 1 of example 360). In some embodiments, a one-step prediction for steady-state Kalman filtering equations can be utilized, as discussed herein.

In some embodiments, with the state estimation and the state covariance, the f value at each time step can be calculated (Step 2 of example 360). In some embodiments, the f value can be calculated using the following equation:

$${f}_{t}^{j} = \frac{{e}^{0.5 ( {y}_{t} - {H}_{j} {x}_{t \vert t - 1}^{j} ) {( {C}_{t}^{j} )}^{- 1} ( {y}_{t} - {H}_{j} {x}_{t \vert t - 1}^{j} )}}{{( 2 π )}^{1 / 2} \sqrt{{C}_{t}^{j}}} \qquad Eq . 11$$

$${C}_{t}^{j} = {H}_{j} {{P}_{t \vert t - 1}^{j} ( {H}_{j} )}^{T} + {R}_{j} .$$

According to some embodiments, with the f value calculated, the model probability can be updated with Markov smoother (Step 3 in example 370). In some embodiments, the model probability for model j at time t is:

$${prob\_ms}_{j} ( t \vert t ) = \frac{{p}_{j} ( t ) {f}_{j} ( t )}{{\sum }_{k = 1}^{4} {p}_{k} ( t ) {f}_{k} ( t )} \qquad Eq . 12$$

$$Where {f}_{j} ( t ) is calculated f value for model j at time t .$$

$${p}_{j} ( t ) = {\sum }_{i = 1}^{4} {π}_{ij} {p}_{i} ( t - 1 \vert t - 1 ) ,$$

Where, πᵢⱼ is the i, jth element in the Markov matrix.

In some embodiments, the EMG index can be defined as the probability for all EMG models, which is 1−the probability for EEG only model, P(model 1). With the EMG index at every time step, engine 200 can calculate the average EMG index in a certain time window. For example, the window size for implementation is 1 second, 500 sample points.

According to some embodiments, the information related to the derivation and compiled EMG index can be stored in database 108, as discussed above.

Accordingly, upon determining the EMG index, the M1 index can be determined. As above, the M1 index construction in Step 316 can be based on the phase-amplitude parameters and BSP (from Step 310) and/or the phase-amplitude parameters, BSP and EMG index (from Step 312).

According to some embodiments, the M1 index can be configured as a data structure with information derived from EEG signals that aims to quantify and enable accurate, real-time monitoring of the depth of anesthesia or sedation. The M1 index integrates various features of the EEG signal to provide a single measure of the patient's level of consciousness. According to some embodiments, the M1 index includes information related to frequency bands, waveform patterns, amplitude, coherence, artifact detection, and the like.

For example, in some embodiments, the M1 index analyzes the frequency content of the EEG signal, considering different frequency bands such as slow, delta, theta, alpha, and beta waves. Changes in the power or dominance of specific frequency bands are indicative of alterations in the patient's level of arousal and consciousness.

In some embodiments, the M1 index incorporates information about the waveform patterns observed in the EEG signal, including burst suppression patterns, spikes, and rhythmic oscillations. These patterns reflect different states of brain activity and are used to assess the depth of anesthesia or sedation.

In some embodiments, the M1 index can provide information related to and/or based on the amplitude of the EEG signal, representing the strength or intensity of neuronal activity, is considered in calculating the BIS index. Higher amplitudes are associated with increased neuronal synchronization and arousal, while lower amplitudes indicate reduced activity and deeper levels of anesthesia or sedation.

In some embodiments, the M1 index can incorporate measures of coherence or synchrony between different regions of the brain, providing insights into the overall connectivity and integration of neural activity.

In some embodiments, the M1 index can include algorithms to identify and minimize the impact of artifacts such as muscle activity, electrocardiogram interference, and electrical noise, which can distort the EEG signal and affect the accuracy of depth-of-anesthesia monitoring.

Accordingly, by combining these features of the EEG, as per the phase-amplitude parameters, BSP and/or EMG index (discussed supra), the M1 index provides a continuous, quantitative measure of the patient's level of consciousness and helps guide anesthesia management to achieve the desired depth of anesthesia while minimizing the risk of POD, among other benefits.

Accordingly, in Step 316, engine 200 can store the M1 index in database 108, as discussed above. Such storage can be in accordance with other patient information obtained, compiled and/or analyzed/determined via the precedent processing in Process 300, and the subsequent processing, via Steps 316-332, discussed infra.

Turning to FIG. 3B, Process 300 continues to provide non-limiting example embodiments for how the compiled M1 index for the patient can be utilized during a procedure in which anesthesia is administered to the patient.

Process 300 continues (from either the compilation and/or storage of the M1 index) with Step 318, where an amount of anesthesia medication for the patient is determined and administered. In some embodiments, the information about the patient (e.g., from Step 302) and/or the M1 index for the patient can be analyzed, whereby a determination as to an initial amount of anesthesia can be performed, which can be administered so that the patient is safety sedated to the proper depth and for the proper time period.

Accordingly, in some embodiments, Step 318 can involve the patient data and/or M1 index being analyzed via engine 200 implementing/executing any type of known or to be known computational analysis technique, algorithm, mechanism or technology. In some embodiments, engine 200 may execute and/or include a specific trained AI/ML model, a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 200 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, and the like. By way of a non-limiting example, engine 200 can implement an XGBoost algorithm for regression and/or classification to analyze the patient data/M1 index, as discussed herein.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define a Neural Network architecture/model,
b. transfer the input data to the neural network model,
c. train the model incrementally,
d. determine the accuracy for a specific number of timesteps,
e. apply the trained model to process the newly-received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

Thus, based on the computational (e.g., AI/ML model-based) analysis engine 200 can determine the proper type and quantity of anesthesia to administer to the patient. This can be displayed on an interface or provided to a healthcare professional enabling them to properly administer the medication.

In Step 320, engine 200 can monitor the patient's vitals while they are sedated. According to some embodiments, as discussed above, during anesthesia, various vital signs can be closely monitored to ensure the patient's safety and well-being throughout the procedure. These vital signs typically include, but are not limited to, EEG (as discussed above), heart rate, blood pressure, respiratory rate, oxygen saturation levels, temperature, and the like.

According to some embodiments, heart rate, measured in beats per minute, provides insight into the cardiovascular system's function and responsiveness to anesthesia. Blood pressure, consisting of systolic and diastolic measurements, indicates the force exerted by blood against the walls of arteries and ensures adequate perfusion to vital organs. Respiratory rate, the number of breaths taken per minute, reflects the adequacy of ventilation and oxygen exchange. Oxygen saturation levels, measured using pulse oximetry, indicate the percentage of hemoglobin saturated with oxygen in the blood, ensuring proper oxygenation. And, temperature monitoring helps detect fluctuations that may indicate physiological stress or complications during anesthesia.

Accordingly, the monitoring of Step 320, which can be performed continuously and/or according to a predetermined time period (e.g., every 10 seconds, for example) can enable prompt intervention based on collected vital signs that contribute to safe anesthesia administration and patient outcomes.

Accordingly, in Step 322, based on the monitoring in Step 320, operative data related to the patient can be collected, and in some embodiments, stored in database 108. Such operative data, as discussed supra, can include, but is not limited to, EEG, heart rate, blood pressure, respiratory rate, oxygen saturation levels, temperature, and the like. Such data can include, but is not limited to, timestamps, metrics/values, identifiers (IDs), and the like. In some embodiments, such data can be collected/stored in association with particular thresholds that correspond to the type of collected operative data. For example, a patient's temperature can be collected as X degrees Fahrenheit, which can be stored in association with a safe range of temperature for, but not limited to, the type of procedure, demographics of the patient, medical history of the patient, duration of the sedation, and the like.

In Step 324, engine 200 can analyze the collected operative data. Such analysis can be based on the M1 index, such that engine 200 can perform a computational analysis to determine whether the observations of the patient, as per their monitored operative data, correspond to the compiled M1 index, which as discussed above, provides a guide for effective anesthesia management. Accordingly, such analysis can be performed via any of the AI/ML analysis techniques discussed above, where the collected operative data can be analyzed based on corresponding M1 index data for the patient.

According to some embodiments, Step 324 can involve the generation of an M1 index for the patient. In such embodiments, an M1 index may not have been generated prior to the commencement of the procedure, such that the M1 index is generated upon operation of Step 324, whereby such generation can be performed in accordance with the steps from FIG. 3A, as discussed supra.

In Step 326, based on the analysis in Step 324, engine 200 can determine whether there has been a threshold satisfying deviation from the M1 index. That is, whether modifications to the currently administered amount (and/or type) of anesthesia are required to ensure avoidance of POD, inter alia. Thus, in a similar manner as discussed above, if the patient's monitored vitals are outside the dynamic range for the M1 index, then alterations to the administered medication may be required. For example, if the heart rate of the patient falls below a threshold rate, then this may indicate that the medication may need to be reduced (e.g., lessen the depth of sedation).

Of course, in some embodiments, a combination of factors of each vitals' data can be accounted for, which can be based on the type of vital failing or falling short of a threshold, a type of medical procedure, duration of the procedure, lapsed time of sedation (or current duration of the procedure), and the like. Such factors, among other directives, can be focused on avoiding POD for the patient. Accordingly, leveraging the AI/ML models discussed above (e.g., CNN, for example) can perform a complex, layered analysis accounting for each type of data input, whereby the determination in Step 326 can be customized for the patient, at that current time, as per the specific circumstances that current patient is currently facing.

As such, when Step 326 results in a determination that no modification to the currently administered anesthesia is required (e.g., each vital or at least a threshold satisfying amount/type of vitals satisfy their respective thresholds, for example), engine 200 can proceed to Step 328. In Step 328, engine 200 can continue the current processing as per the proceeding steps, and revert back to Step 320 to continue the monitoring of the patient until the end of the procedure. In some embodiments, the determined information from Step 326, and corresponding data from which the determination of Step 326 was based, can be stored in database 108, and used to update the M1 index for the patient. As above, the M1 index is dynamically updated with real-time patient data, therefore, the M1 index can be updated via similar steps discussed above to reflect current values for the patient. For example, the current vitals can serve as the data (for Step 302), where updated M1 values can be compiled and used to update the patient's M1 index (which can occur while continued monitoring is performed).

When the determination in Step 326 indicates that the anesthesia amount (and/or) type is determined to require an update, engine 200 can proceed to Step 330. In Step 330, engine 200 can perform a determination of the anesthesia values based on, but not limited to, a current stage of the procedure, collected operative data of the patient and/or the M1 index of the patient. Such determination, which can be performed via the AI/ML analysis and determinations discussed above, can be performed in a similar manner as discussed above respective to Step 318. Such compiled anesthesia update values can be stored in database 108.

And, in Step 332, engine 200 can compile and communicate a message for modification of the anesthesia. Such message, which can be an electronic message that is displayable within a user interface (UI) or graphical user interface (GUI) of a display screen of a device (e.g., user device and/or screen in emergency room (ER), for example), can provide the anesthesiologist (or other medical professional) with instructions for how to modify the current dosage to the recommended dosage. In some embodiments, such message can include computer-executable instructions that can automate the dosage change, whereby user input may not be required.

Accordingly, as indicated in FIG. 3B, upon performance of Step 332, engine 200 can proceed back to Step 320 to monitor the updated anesthesia dosage for the patient until the completion of the medical procedure, and upon the raising of the patient from sedation. This can ensure that POD is avoided.

Thus, as discussed above, the disclosed systems and methods, as evidenced from the above discussion, provides an advanced, dynamically applied medical assessment framework that ensures a patient's perioperative safety.

Moreover, as discussed above, by way of non-limiting examples, when patients are unconscious under propofol or sevoflurane anesthesia, their EEG shows profound, stereotyped slow (0.1 to 1 Hz) and frontal alpha (8 to 12 Hz) oscillations that reflect periodic silencing of cortical activity and disruption of prefrontal thalamocortical processing, respectively. This pattern is visible in older patients, but the size of the EEG declines significantly in old age likely a consequence of cortical atrophy in the prefrontal cortical areas that generate anesthesia-induced EEG oscillations. This significant decline in EEG power, and alpha power in particular, makes the anesthesia-induced EEG in older patients resemble an “awake” EEG, leading to, when using conventional mechanisms, erroneously high BIS readings. In turn, the only way to lower the erroneously high BIS number is to increase the anesthetic dose and place the patient into “burst suppression.” This is a major flaw in the design of the BIS that leads to suboptimal care in a significant proportion of surgical patients who are elderly.

For example, FIG. 3H and FIG. 3I shows how each compares in performance for the tracked surgical journey for two (2) example subjects: one with delirium and one without delirium. The patients are older patients (e.g., age >60) with weak alpha energy. As in FIG. 3I, the M1 index is capable of showing different patterns between delirium subjects (line 426) and no-delirium subjects (line 424), suggesting that the patient who ultimately became delirious was too deeply anesthetized, and that they could have received lower levels of anesthetic that could have reduced their risk of POD. Conversely, as in FIG. 3H, the BIS is not able to tell the difference between delirium patients (line 422) and no-delirium patients (line 420), and, moreover, provides values suggesting that both of these patients are too lightly anesthetized, suggesting that higher levels of anesthesia could be administered. Indeed, as in FIG. 3I, these cases illustrate how the M1 index includes functionality for indicating over-sedation, further providing an indication that the anesthetic concentration could be reduced, potentially leading to a reduced risk of delirium.

Moreover, the non-limiting example in FIG. 3K depicts how the disclosed M1 index outperforms current technologies (e.g., BIS index) in estimating individual patient's consciousness and/or unconsciousness states. Examples of the M1 index improvement over conventional methods (e.g. as compared to BIS), is provided in FIG. 3J and FIG. 3M-3O. The functionality related to the disclosed M1 index's capabilities related to improved state of consciousness is discussed above.

As depicted in FIG. 3K, the M1 index is an improvement to the BIS index. As depicted in FIG. 3K, M1 index is significantly more accurate, accounting for both the age of patients and the specific anesthetic in use. As illustrated in FIG. 3K, via a rank-sum testing, results showed that the M1 algorithm achieved a median accuracy of 0.91 [IQR: 0.85, 0.96], which significantly outperforms the BIS's median accuracy of 0.50 [IQR: 0.43, 0.57] in patients aged 60 or older (p=9.4e−14<0.01; FIG. 7, left). The disclosed M1 index also outperforms BIS in patients 60 years old or younger (p=2.9e−11<0.01).

Moreover, the above disclosed BSP calculation steps can involve, but are not limited to: calculating surpression_state from the EEG spectrogram, wherein the input for this logistic regression model x_t is an n*1 vector, representing the spectrogram of the observation signal at time t. The output y_t is a scaler 0 to 1, representing the suppression state at time t. y_t=1 means it is suppression at time t. y_t=0 means it is burst at time t.

In some embodiments, after init parameters and loading the data, eeg_spectrogram is a 257*t matrix, that represents the EEG spectrogram in t seconds. 257 is based on sampling rate=512 and time window=1 second. From prior knowledge, only the low-frequency component of the spectrogram is needed for burst suppression detection. Here I use frequency from 0˜21. The purpose of this step above is to reduce the computational cost for both training and testing and reduce the model variance.

Next, the real-time testing procedure is the following. Let's note the input EEG spectrogram at t second is x_t, the output y_t_pred is:

$${y}_{t}^{pred} = 1 - 1 / ( 1 + {e}^{{β}_{0} + β {x}_{t}} ) , \qquad Eq . 13$$

beta_0 and beta are parameters fitted from training data. After that, set a threshold=0.5 to transfer this y_t_pred to_{suppression_state}. In some embodiments, the threshold is, for example, 0.5 per the definition of Burst and Suppression event.

$$if prob\_pred \_logisitc [ 0 , i ] < 0.5 : supression\_state [ 0 , i ] = 1. \qquad Eq . 14$$

The second part is to calculate burst suppression probability from surpression_state. Here the input b_t is a binary value 0 or 1. b_t=1 means it is suppression at time t. b_t=0 means it is burst at time t. The one-step prediction is the following:

$${σ}_{t \vert t - 1}^{2} = {σ}_{t - 1 \vert t - 1}^{2} + {σ}^{2} \qquad Eq . 15$$

$${x}_{t} = {x}_{t - 1} + {σ}^{2} ( {b}_{i} {p}_{t - 1} )$$

$${σ}_{t \vert t - 1}^{2} = 1 / ( \frac{1}{{σ}_{t \vert t - 1}^{2}} + \frac{1}{{p}_{t} ( 1 - {p}_{t} )} )$$

$${p}_{t} = {e}^{{x}_{t}} / ( 1 + {e}^{{x}_{t}} ) .$$

The state covariance sigma and the init state x_0 are parameters in this model, which can be trained using the EM algorithm. For real-time implementation, in some embodiments, sigma may not be updated using EM algorithm, as fix parameter may be used

Thus, the disclosed M1 index mechanisms, discussed supra, employs EEG markers of anesthesia-induced brain states that remain accurate for all patients, even older patients and those with dementia. Indeed, the disclosed mechanisms and functionality are capable of being used with EEG features to predict POD (and/or other side effects, medical conditions and/or mental health issues/concerns), manage a patient's level of consciousness, and/or detect when patients may have lower anesthetic requirements. An example of this, as compared to BIS, is provided in FIG. 3L.

According to some embodiments, the constructed simulated EEG waveforms employ the state space models to mimicking the key changes in the EEG that occur during aging or dementia, while preserving those that indicate the patient's state of consciousness/anesthesia. To demonstrate how well M1 monitors old or demented patients compared to BIS, four sets of EEG data are characterized: 1) “Real Young:” 10 young patients (age <35) who received general anesthesia during surgery; 2) “Real Old:” 10 old patients (age >65) who received general anesthesia during surgery; 3) “Digital Aged:” 10 digitally-aged surrogates of the “Real Young” patients; 4) “Digital Dementia:” 10 digitally-demented surrogates of the “Real Young” patients. Both M1 index and BIS values are calculated for each subject in each group. In some embodiments, a validated, highly-accurate open-source BIS emulator called ‘OpeniBIS’ can be used to calcuate BIS values. As expected, when BIS was applied to the “Real Young” group, on average the values were largely within the recommended range of 40 to 60 to maintain appropriate anesthesia/unconsciousness during general anesthesia (FIG. 3L). However, BIS showed inaccurate, elevated readings under aging and dementia: 70% of the “Real Old” patients and all of the “Digital Aged” and “Digital Dementia” patients had high values above the recommended range of 40 to 60 (FIG. 3L), erroneously implying that these patients were not sufficiently anesthetized and that higher levels of anesthesia would need to be administered. In contrast, when using the M1 index, all patients' values were within the correct range (FIG. 3L). Thus, the M1 index was much more accurate in older and demented patients than the BIS algorithm.

A further example of the capabilities of the disclosed mechanisms and functionalities using the EEG to predict POD (and/or other side effects, medical conditions and/or mental health issues/concerns), manage a patient's level of consciousness, and/or detect when patients may have lower anesthetic requirements, this time in a cohort of patients, is illustrated in FIG. 3N and FIG. 3O. Among other due diligence activities, the inventors studied n=71 subjects receiving general anesthesia for surgery. In this group, 14 developed POD, while 57 did not. In some embodiments, it was estimated that the average trajectory of M1 index for POD versus non-delirious patients, time normalized for each case to allow the trajectories to be comparable. It was found that the M1 index was significantly lower in the patients who developed POD compared to those who did not (Wilcoxon rank sum test, p<0.05) (FIG. 3N, showing no-delirium patients at line 430 and delirium patients at line 432). For the same group, the BIS values showed no significant difference between groups (Wilcoxon rank sum test, p=. 81) (FIG. 3O, showing no-delirium patients at line 440 and delirium patients at line 442). In agreement with the example presented supra, the BIS values were above the range corresponding to general anesthesia, suggesting that these patients would require administration of higher levels of anesthesia. In both this example and the previous one, BIS provides incorrect and opposite guidance for the most vulnerable patients who are older, who may have ADRD, and who ultimately go on to develop POD.

In a further example, in FIG. 3M three representative patients are presented from the n=14 patients described supra who developed delirium. Each of these patients, as indicated in the upper portion of each figure, have different medical diagnoses indicated by the ICD code, are having different procedures performed as indicated by their CPT codes, have different ages, and different genders. As above, it is estimated the average trajectory of the M1 index for each patient, time normalized to allow the trajectories to be comparable. It can be found that the M1 index (lighter line) was substantially lower compared to BIS values (darker line) throughout the majority of the case. Thus, in each of these individual patients who developed POD, each with different medical histories, procedures, ages, and genders, the M1 index indicates that they could have received lower amounts of anesthesia, which might have reduced their risk of delirium. Meanwhile, BIS provided misleading information that these patients were too lightly anesthetized and that the anesthetic level should be increased.

According to some embodiments, the disclosed systems and methods, as discussed supra (e.g., the steps of Process 300) can be implemented within a closed-loop system to effectuate an automatic and dynamic, real-time control and management of a patient (e.g., biometrics, and/or other characteristics) during a procedure to curate and apply real-time anesthesia amounts to not only keep the patient safe during the procedure, but also prevent and/or mitigate the onset of any type of side effect, medical condition and/or mental health condition. As discussed above and in more detail herein, determined values, via the M1 index (as an example only, other control signals meeting the specifications in this standard can be used), for example, can be determined in real-time during a procedure and applied to ensure the safety and safe recovery from the procedure and anesthesia post-op.

In some embodiments, the closed-loop control system may be employed to increase compliance and accuracy for which a desired target anesthetic level may be maintained during anesthesia. The closed loop system can be used to monitor and adjust the level of various drugs, including but not limited to anesthetics, pain management medication, and/or muscle control medication. This closed loop system would include the monitoring components described above, e.g., an EEG sensor, an acquisition unit, and/or processors, to analyze the EEG sensor information and produce monitoring variables such as the M1 index, along with additional processing units to calculate feedback control information to regulate the value of any of the anesthetic variables mentioned above, such as M1, phase-amplitude modulation, AMI, and/or SMI, blood pressure, and/or indicators of nociception, pain, or muscle relaxation, based on any number of established control algorithms. In some embodiments, the closed-loop system would include an interface module providing bi-directional communication between the feedback control processing unit and at least one infusion pump, anesthetic machine administering inhaled anesthetics, or other drug delivery system. The bi-directional communication module would enable the feedback control processing unit to provide commands to the infusion pump to establish the desired infusion rate of the drug and also to receive pertinent information about the operational status of the pump including verification of the commands received, the commands executed, measured flow rates, error states such as pump occlusion or other errors in flow rate, remaining syringe or fluid volume or other variables relevant to the operational status of the pump.

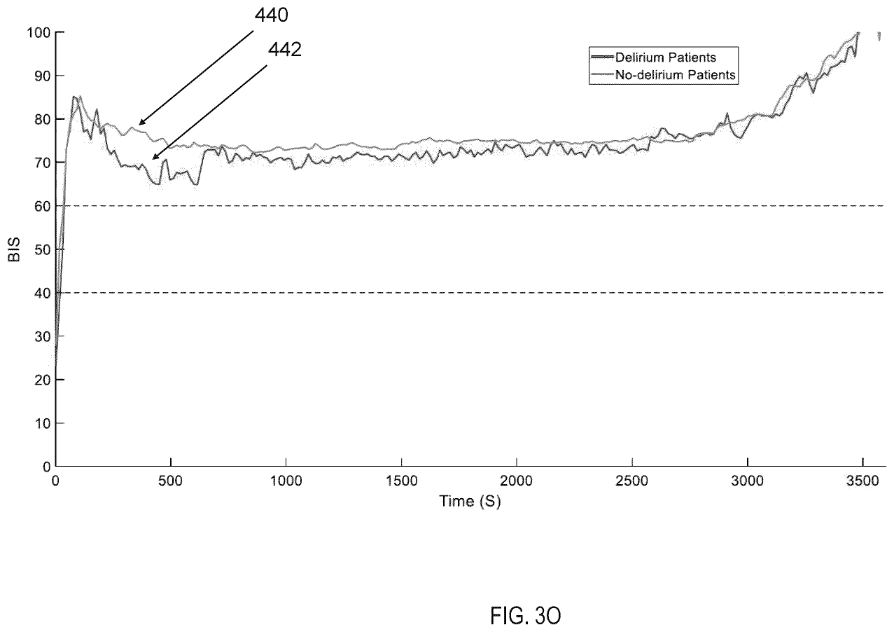

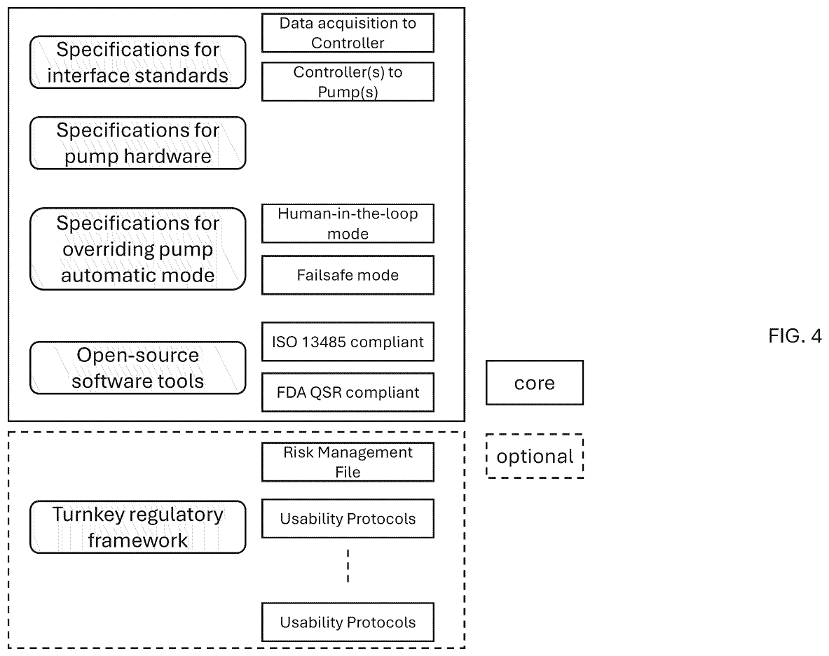

Although closed-loop controllers have been long sought-after in anesthesiology, critical care, emergency medicine, combat casualty or disaster management, or related medical fields, at least three critical issues have impeded the development of commercial closed-loop control systems. First, particularly in the case of closed-loop control of general anesthesia and sedation, appropriate control variables that provide accurate assessments of a patient's brain physiological state that are linked to desired intra-operative requirements and post-operative outcomes have been lacking. Second, establishing regulatory approval for a closed-loop system is regarded as challenging since the regulatory burden of an infusion pump or other drug delivery devices is compounded by the further regulatory burdens of a physiological monitoring system as well as a closed-loop control system that integrates the operation of the pump and the monitoring system. Standards, systems, and methods that can simplify or streamline risk assessment and risk management of the combined closed-loop system by compartmentalizing the risk assessment and risk management required of any manufacturer of any component of the overall system would thus be highly advantageous in reducing the regulatory burden of a closed-loop system. Third, assignment of responsibility and legal liability for any performance failures in the system is potentially ambiguous and would constitute an impediment to commercial progress as commercial entities might not be able to assess the financial risk or liability for product failures in the context of such ambiguity. As presented earlier here, described herein are novel methods for quantifying anesthesia-induced brain states that can provide accurate assessments of a patient's brain physiological state that are linked to desired intra-operative requirements and post-operative outcomes, thus substantially addressing the first problem. In the following novel systems and methods are disclosed to compartmentalize regulatory risk among the constituent components of infusion pumps or other drug delivery systems, physiological monitoring systems, and closed-loop controllers in order to address the second problem. Solutions addressing this problem can substantially alleviate the liability concerns described above, as responsibility and legal liability would be largely assigned according to the boundaries of the regulatory risk defined by these systems and methods and standards that may be established based on those systems and methods. FIG. 4 illustrates an exemplary embodiment of an overview of the standard, showing core components and optional components that may provide regulatory support for manufacturers adopting the standard. A pump command interface specification that allows interoperability between pumps.

The ability of a closed-loop control system to operate safely and effectively with a variety of infusion pumps is a desirable feature to enable widespread adoption of monitoring and closed-loop control systems. At a given moment of time, a healthcare facility might have an assortment of drug infusion or delivery systems installed across their clinical spaces of different makes and models that may have significant remaining service life making it unnecessary or wasteful to replace en masse with a specific system dedicated to closed-loop control. Moreover, the closed-loop control applications required in any given circumstance may vary by the setting, the clinical specialization, and even by patient. These varying needs require flexible configurations among monitors, closed-loop controllers, and infusion or drug delivery systems. At the same time, safety and efficacy cannot be compromised; thus systems that facilitate interoperability among a infusion pumps and other drug delivery systems to maintain relevant parameters for safe and effective operation are highly desirable.

To address this problem, an interface that allows a closed-loop controller to interact with a specific infusion pump or other drug delivery device could have two modules: 1) The closed-loop controller could have a module that specifies a) the command outputs from the controller (e.g., desired flow rate), b) status inputs to be received from the pump (e.g., acknowledgement of command receipt, measured flow rate), and c) defines a failsafe mode of open-loop operation that the infusion pump executes when communications with the closed-loop controller are interrupted or when closed-loop control is deemed to be non-functional (e.g., loss of physiological sensor fidelity); 2) There would be a device driver written for a specific pump that a) translates the command outputs received from the controller into the actions required by the pump to execute the command, b) obtains the required status information from the pump and reports that information to the controller, c) defines a protocol for manual override of the closed-loop controller by the pump (e.g., if a clinician interacts with the manual controls of the pump to specify an infusion rate or other mode of action).

This interface could be constructed by any number of approaches known in the art including but not limited to:

### Communication Protocol

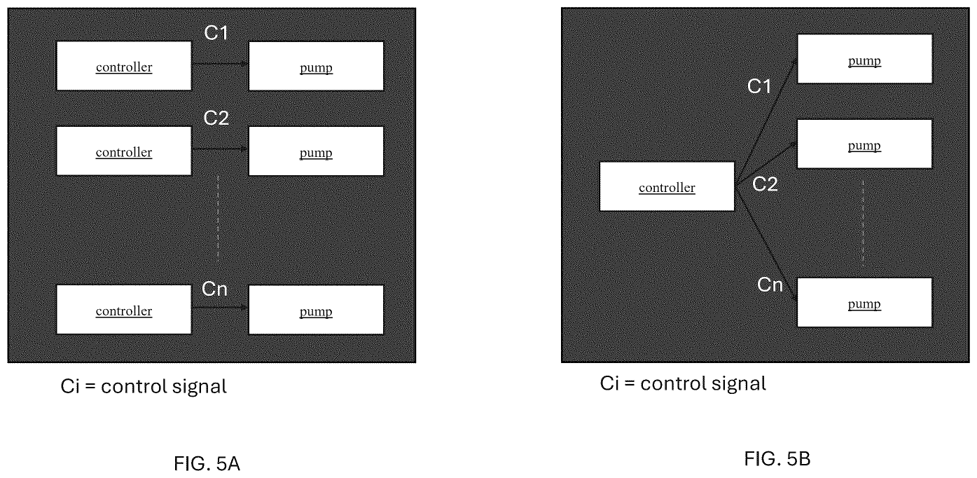

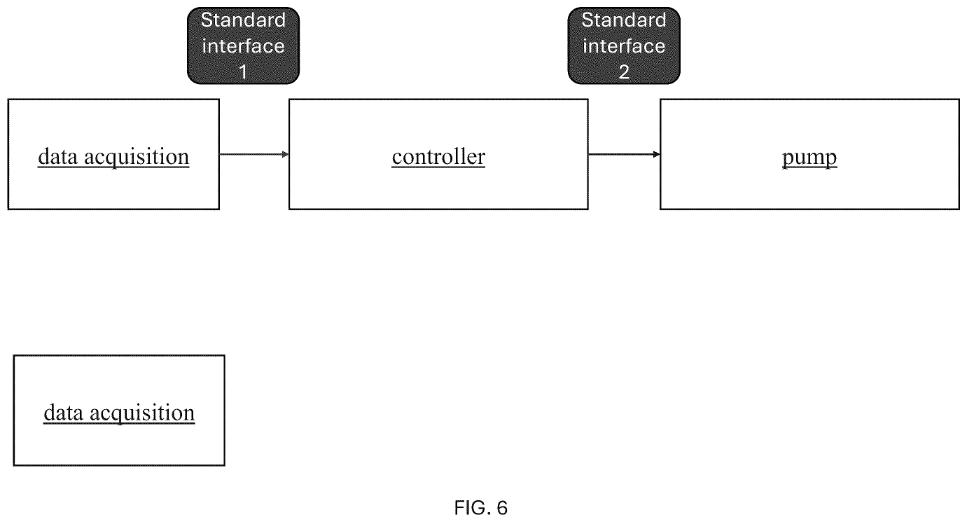

In some embodiments, the system utilizes a custom communication protocol to facilitate communication between one or more pumps for drug delivery and control systems either via serial communication or wireless communication. This protocol supports multi-device networks, allowing for the simultaneous control and monitoring of multiple pumps. FIGS. 5A and 5B illustrate various exemplary embodiments of communication protocol for communication between one or more controllers and one or more pumps.

### Command Structure

In some embodiments, the command structure of the invention is based on custom command protocol. Each command is formatted in a standardized syntax, enabling the setting of operational parameters such as flow rates, pressure levels, and operational states (e.g., start, stop, pause).

### Data Format and Units

In some embodiments, the data format utilizes standardized units for all operational parameters. For example, flow rates are expressed in milliliters per minute (mL/min) or microliters per minute (μL/min), ensuring consistency across different devices.

### Addressing and Device Identification

In some embodiments, the invention includes a unique addressing scheme for each pump within a network. This allows for individual control and status monitoring, facilitating the management of complex systems with multiple pumps.

### Error Handling and Feedback

In some embodiments, the interface provides mechanisms for error detection and handling. Commands are acknowledged with specific responses (e.g., “OK” for successful execution), and error messages are standardized to facilitate troubleshooting.

### Settings and Configuration

The specification allows for the configuration of various pump settings, including:
Maximum and Minimum Flow Rates: Commands to set these parameters ensure pumps operate within their specified ranges.
Pressure Levels: Commands to adjust and monitor pressure levels.
   Integration with External Systems

The interface is designed to be compatible with external systems, such as electronic medical records (EMR) in medical applications, ensuring secure and efficient data exchange.

Over-ride specifications inclusive of UI/UX properties can allow switch-over to manual or open loop operation when feedback sensing fails (e.g., human in the loop, failsafe mode).

The failsafe mode of operation and the manual override conditions may be implemented according to any number of approaches known to the art.

In some embodiments, override specifications are design parameters that allow a system to switch from automated to manual control when needed. In some embodiments, key details include trigger conditions (e.g., specific thresholds or error states that initiate override), Control transfer (e.g., mechanisms to smoothly transition control to the operator), Manual interface (e.g., dedicated controls for human operation), and system status indicators (e.g., clear visual/auditory cues showing override is active). These specifications enable override by providing a pre-planned pathway to shift control, ensuring the system remains operable even if automated functions fail.

In some embodiments, the user interface for override functionality can incorporate prominent override activation controls, clear status indicators showing current operating mode, simplified manual control schemes, high-contrast visual design for critical information, auditory and haptic feedback for important actions, and/or contextual help and guidance for manual operation. These properties ensure operators can quickly understand system status and effectively take control when needed.

In some embodiments, feedback sensing failure can be determined through data validation checks on sensor inputs, comparison of multiple redundant sensors, monitoring for frozen or stuck values, detection of values outside expected ranges, and/or watchdog timers to catch non-responsive sensors. In some embodiments, when failure is detected, the system should:
1. Alert operators of the issue
2. Attempt to use backup sensors if available
3. Enter a degraded operating mode with limited functionality
4. Initiate override procedures if human intervention is required

In human-in-the-loop mode of operation:
1. The system transfers primary control to a human operator
2. Automated functions shift to an advisory role, providing suggestions
3. Safety-critical automated functions may remain active as a backstop
4. The interface adapts to prominently display manual controls and key system data
5. Operators can selectively re-engage automated functions as needed

While human control is primary in this mode, some automated operations can still be utilized for safety interlocks to prevent dangerous actions, data logging and analysis to aid decision-making, automated diagnostics to help troubleshoot issues, and/or low-level control loops for basic system stability.

A pump physical specification inclusive of all dynamics that can be used to specify the controller properties that guarantee stability.

Rigorous analysis and verification of performance characteristics such as the stability, impulse response, or frequency response, are essential for guaranteeing safety and efficacy of any closed-loop control system. A comprehensive analysis of such performance characteristics requires a combination of analytical, computational, and real-world experimental approaches. In envisioning scenarios as described earlier in which a closed-loop controller may be combined with a variety of potential infusion pump or drug delivery systems, systems and methods are needed to facilitate such analyses consistent with the highest standards of engineering practice and regulatory requirements. Such systems and methods to support performance analysis at the analytical, computation, and experimental levels is described below.

Analytical methods: Characterization of the performance of a complete closed-loop system must encompass the dynamics of the complete system inclusive of both the closed-loop controller as well as the dynamics of the pump and any attached mechanical elements such as syringes, cartridges, intravenous tubing or other similar items in any number of configurations that may be enabled in clinical practice. Accordingly, an analytical description must be provided for the dynamics of a) the closed-loop control system and b) the pump and any attached mechanical elements. The analytical description can take any number of forms consistent with control engineering practice, including but not limited to differential equations, frequency domain methods such as transfer functions in the Lapalace or Fourier domain, impulse response functions, or state space models in the form of a linear quadratic regulator. In one embodiment, the dynamics of the closed-loop controller could be described in a number of equivalent or comparable forms, (e.g., a state space model and/or a transfer function) in which case a compatible description of the dynamics of an infusion pump to be used with this controller would be provided in a compatible analytical framework (e.g., a state space model and/or a transfer function, respectively). With these descriptions, the combined dynamics of the closed-loop controller and the infusion pump or drug delivery device could be analyzed for stability, frequency response, impulse response, and other characteristics of interest.

Computational methods: Given the analytical descriptions of the closed-loop controller and the infusion pump or drug delivery device, computational modules could be constructed to implement those dynamics in silico to verify the stability, frequency response, impulse response, and other characteristics of interest. Any number of computational approaches appropriate to the analytical framework could be selected to accurately and faithfully calculate system dynamics, including but not limited to methods described below.

Experimental methods: For a given closed-loop controller, a series of experiments may be defined in which a sequence of commands are specified to interrogate different performance features or requirements of the system (e.g., the step response to characterize the slew rate of the system). These experiments could be run both in silico using the computational methods described above as well as in a physical laboratory setting in which flow rates or fluid volumes are measured, to verify consistency between the computation results and the laboratory experimental results.

For a given closed-loop controller, software tools could be developed to facilitate in silico testing for any infusion pump or drug delivery system. For instance, such tools could include libraries that implement the dynamics of the closed-loop controller, the controller output commands, the infusion pump driver, the dynamics of the infusion pump and any mechanical accessories, such that the full system can be characterized in silico in any desired scenario to establish regulatory compliance.

In some embodiments, specifications for pump stability can include:

### Pump Characteristics

Pump performance curves (head vs. flow)
Net positive suction head (NPSH) requirements
Operating speed range
Impeller design and number of vanes

### System Dynamics

Natural frequencies of the pump and piping system
Damping characteristics
Inertia of rotating components

### Flow Conditions

Flow rate range
Suction and discharge pressure ranges
Fluid properties (viscosity, density, temperature)

### Controller Specifications

Response time
Gain settings
Feedback mechanisms

### Key Considerations

Resonance Avoidance: A critical aspect of ensuring stability is to avoid resonance between the pump's operating frequencies and the system's natural frequencies. This requires a detailed lateral structural analysis and potentially a rotordynamic stability analysis for high-energy or high-speed pumps.

Dynamic Analysis: The ANSI/HI 9.6.8 Rotodynamic Pumps-Guidelines for Dynamics of Pumping Machinery standard provides guidance on determining appropriate levels of dynamic analysis. This can include forced response bending stress analysis and rotordynamic stability analysis for more complex systems.

Vortex Formation: Physical modeling is still considered the most reliable method for predicting vortex formation and strength, which can significantly impact pump stability. While computational fluid dynamics (CFD) can provide insights into flow patterns, it is not yet recognized by industry standards for predicting inlet flow patterns or compliance with stability criteria.

Operating Range: Specifying a wide stable operating range is crucial. This should include considerations for part-load operation, as instabilities often occur at lower flow rates.

Cavitation Prevention: Ensuring adequate NPSH throughout the operating range is essential to prevent cavitation-induced instabilities.

Motor and Drive Characteristics: For systems with variable speed drives, the motor and drive specifications must be carefully matched to the pump characteristics to avoid instabilities across the speed range.

Open source in-silico modeling software tools can be used that facilitate rigorous computer in the loop testing that would satisfy FDA and CE regulatory requirements.

In some embodiments, a software to implement the specifications of the standards can be developed and opensource for others to use. The software will be developed following FDA Design Control guideline and under ISO 13485 [cite] to meet FDA and CE regulatory requirements.

In some embodiments, a regulatory framework can be employed that clearly defines the regulatory responsibilities of all relevant stakeholders (pump manufacturers, monitoring company, interface company, physician, hospital) so as to absolve pump companies of any additional liability beyond their baseline liability to maintain the basic operating characteristics of the pump. In some embodiments, turnkey components can include:
1. Risk Management Files: Comprehensive risk management templates and guidelines, covering risk analysis, evaluation, control, and residual risk assessment. The risk management system for integrating brain monitors with anesthesia delivery pumps comprises a risk management plan that defines the objectives, scope, and criteria specific to hardware, software, and human factors associated with the pumps. The system includes hazard identification and analysis, addressing mechanical failures, power supply failures, software bugs, cybersecurity risks, user interface errors, training and usage errors, as well as environmental risks such as temperature extremes and electromagnetic interference. The risk evaluation process involves assessing the severity of each identified hazard and the probability of its occurrence. The system further includes risk control measures, incorporating preventive actions to minimize or eliminate identified risks and mitigation measures to reduce the impact of potential risk scenarios. Residual risk evaluation is conducted to assess the remaining risks post-mitigation, ensuring that they are within acceptable limits. Additionally, the system includes a comprehensive risk management report that documents the identified risks, control measures, and the rationale for the acceptance of any residual risks, along with a continuous monitoring plan to oversee risk-related data on an ongoing basis.
2. Usability Protocols: Ready-to-use protocols and templates for usability testing, ensuring compliance with regulatory requirements for human factors engineering.
3. Software Development Lifecycle Documentation: Documentation templates and guidelines for the entire software development process, ensuring alignment with ISO 13485 and FDA QSR requirements.
4. Design History File (DHF) Templates: A complete set of templates for the Design History File, including design plans, reviews, and verification/validation reports.
5. Pre-Market Submission Support Materials: Templates and guidelines for compiling pre-market submissions such as 510(k) or PMA, tailored for systems integrating your standard.
6. Supplier Qualification Protocols: Protocols for qualifying suppliers, ensuring that all components and software meet regulatory standards.
7. Change Management Procedures: Standard operating procedures (SOPs) and templates for managing changes in the design or process, maintaining compliance and traceability.
8. Post-Market Surveillance Plans: Predefined plans and protocols for post-market monitoring, including complaint handling and Corrective and Preventive Action (CAPA) processes.
9. Quality Agreements: Pre-drafted quality agreements outlining the responsibilities and obligations of stakeholders involved in the integrated system's lifecycle.

Turning to FIG. 10A, disclosed is Process 1000 for which an M1 index can be utilized to identify, manage, control and/or manipulate a consciousness level of a patient during an operation.

According to some embodiments, as discussed herein, an M1 index can be a representation of the real-time level of consciousness of a patient undergoing anesthesia and can be calculated by analyzing frontal EEG signals. In some embodiments, the M1 index can be and/or maintain a continuous variable(s), which can range from 0-100, providing similar interpretation for users familiar with the indices of existing commercial EEG products. However, the M1 index value is calculated using a fundamentally different approach than such conventional approaches. That, as discussed herein, the disclosed M1 index calculation/determination involves, inter alia, state-space modeling of discrete brain states identified using specific EEG features, which can enable predictive, reactive and/or some combination thereof, of EEG values to ensure safe and healthy consciousness levels are maintained throughout (and after) a medical procedure.

By way of example, FIG. 10B depicts an example range of M1 index values for specific types and/or stages of anesthesia. When a patient is under anesthesia, their level of consciousness is typically reduced to the point where they are unaware of their surroundings and do not experience sensations or pain. This state of altered consciousness can be achieved through the administration of anesthetic agents that depress the central nervous system. Depending on the type and depth of anesthesia, the patient may be in a range from light sedation, where they may have minimal awareness or respond to stimuli, to deep general anesthesia, where they are completely unconscious and unresponsive. During this period, the patient's physiological functions can be closely monitored to ensure stability and safety.

As per the disclosure herein, the disclosed M1 index's implementation and usage can create an optimal environment where the patient remains entirely unaware and unaffected by the surgical or procedural stimuli, while minimizing any potential side effects or complications.

According to some embodiments, Step 1002 of Process 1000 can be performed by identification module 202 of assessment engine 200; Steps 1004-1008 can be performed by determination module 204; Steps 1010-1012 can be performed by control module 208; and Step 1014 can be performed by monitoring module 206.

In some embodiments, Process 1000 begins with Step 1002, where information related to, but not limited to, a patient and/or medical procedure are collected (or identified). In a similar manner as discussed above in relation to Process 300, such information can correspond to, but not be limited to, a patient's information (e.g., baseline and/or intraoperative information), anesthesia levels, EEG values/levels, type of procedure, and the like, or some combination thereof. Thus, for example, in Step 1002, a patient is under anesthesia as a procedure has commenced (as in FIG. 3B, discussed supra).

In Step 1004, engine 200 can determine EEG signals for the patient. In some embodiments, the identification, analysis and such determination can be performed in a similar manner as discussed above in relation to Process 300.

According to some embodiments, during a procedure involving anesthesia, several types of EEG signals can be monitored to assess and maintain the appropriate level of consciousness. In some embodiments, raw EEG signals can provide a direct recording of the brain's electrical activity, displaying different frequency bands such as slow, delta, theta, alpha, and beta waves. Analyzing these signals helps gauge the depth of anesthesia by revealing changes in brain wave patterns. For example, slow/delta waves are associated with deep sleep or unconsciousness, while alpha and beta waves are indicative of lighter states or wakefulness. Additionally, processed EEG data as per the M1 index discussed above and in more detail herein, can provide a more refined assessment of consciousness levels by providing a single numerical value that reflects overall brain activity and sedation depth. Monitoring these EEG signals allows anesthesiologists to adjust the anesthetic agents appropriately, ensuring the patient remains in the desired state of consciousness throughout the procedure.

According to some embodiments, frontal EEG signals can be identified and analyzed, which refer to the electrical activity recorded from the frontal lobes of the brain. These signals are obtained by placing electrodes on the scalp in positions corresponding to the frontal regions. The frontal lobes can be involved in higher cognitive functions such as decision-making, problem-solving, and emotional regulation, so monitoring their activity can provide valuable insights into a patient's mental state and brain function.

In the context of anesthesia and medical procedures, frontal EEG signals can be particularly useful. Such signals can help assess the effects of anesthesia on the frontal lobe's activity, which is crucial for evaluating the patient's level of consciousness and cognitive responsiveness. For example, changes in the frequency and amplitude of frontal EEG waves can indicate different states of sedation or unconsciousness. During deep anesthesia, the frontal EEG can show predominance of slow/delta waves, while lighter sedation might reveal more mixed activity with higher frequency alpha or beta waves.

Overall, monitoring frontal EEG signals allows for a more targeted approach to managing anesthesia, as the frontal lobes play a key role in awareness and cognitive function. This helps ensure that the patient remains in the appropriate state of consciousness for the duration of the procedure.

In Step 1006, engine 200 can execute state-space modelling based on the EEG signals. Such state-space modelling, in some embodiments, can involve execution of any of the AI/ML models discussed above.

According to some embodiments, state-space modeling can be effectively applied to EEG signals to analyze and interpret brain activity by providing a structured framework for understanding the dynamic nature of neural processes. In state-space modeling, the brain's electrical activity is represented using a set of state variables and equations that describe how these variables evolve over time. This approach can be particularly useful for several reasons, which can involve dynamic analysis, noise reduction, temporal patterns, prediction and control, and dimensionality reduction, among other benefits.

For example, state-space models capture the temporal dynamics of EEG signals, allowing for the examination of how brain activity changes over time. This is important for understanding transient states and responses to stimuli or interventions.

In another example, EEG signals are often noisy and can be influenced by various artifacts. State-space modeling helps in filtering out noise and separating the true neural signals from artifacts by incorporating noise models into the state-space framework.

Moreover, by using state-space models, engine 200 can identify and characterize temporal patterns in EEG data, such as oscillatory rhythms or event-related potentials. This helps in understanding how different brain regions interact and how neural activity patterns are associated with different cognitive states.

Furthermore, state-space models allow for the prediction of future EEG signal patterns based on current and past data. This can be useful for anticipating changes in brain activity and for controlling or adjusting interventions, such as anesthesia, in real-time.

And, EEG data can be high-dimensional and complex. State-space models help reduce this complexity by summarizing the brain's activity in terms of a smaller set of state variables, making it easier to interpret and analyze the data.

In practical applications, state-space models can be used to track changes in brain states during different cognitive tasks or medical procedures, assess the impact of interventions, and improve the precision of neurofeedback or brain-computer interface systems. Overall, state-space modeling provides a powerful tool for extracting meaningful information from EEG signals and understanding the underlying neural dynamics.

According to some embodiments, as understood by those of skill in the art, a state-space model can be a representation, as an executable data structure and/or set of executable data structures in terms of its states, inputs, outputs, and dynamic equations. Such executable models can be expressed in a set of first-order differential (or difference) equations, capturing the evolution of the system's state vector over time. The state vector, typically denoted as x(t), encapsulates all the necessary information about the system's current status, enabling the prediction of future behavior. The model consists of two primary equations: the state equation and the output equation.

In some embodiments, a state equation is defined as:

$${x}_{t} = {Ax}_{t - 1} + {q}_{t} , {q}_{t} \sim N ( 0 , Q )$$

where xₜ is the state variable, A is the state transition matrix describing the system dynamics, qₜ represents the process noise, and Q is the transition covariance.

In some embodiments, an output equation is given by:

$${y}_{t} = {Hx}_{t} + {r}_{t} , {r}_{t} \sim N ( 0 , R )$$

where yₜ represents the observation signal, H is the observation matrix, rₜ denotes the observation noise, and R is the observation covariance.

As discussed above, and depicted in the above equations, the disclosed framework implements a linear state-space model, which can be utilized for control theory and signal processing applications. The state evolution equation describes how the system state xₜ progresses over time, where the state transition matrix A governs the deterministic dynamics of the system, and the process noise qₜ accounts for uncertainties and disturbances that affect the state evolution. The process noise follows a multivariate normal distribution with zero mean and covariance matrix Q, which characterizes the magnitude and correlation structure of the system uncertainties.

The observation equation establishes the relationship between the true system state and the measured observations yₜ. The observation matrix H defines how the internal states map to the observable quantities, while the observation noise rₜ represents measurement errors and sensor limitations. Similar to the process noise, the observation noise is modeled as zero-mean Gaussian with covariance matrix R, which quantifies the measurement accuracy and potential correlations between different sensor channels.

This formulation provides the foundation for optimal state estimation algorithms such as the Kalman filter, which recursively estimates the system state by optimally combining the predicted state evolution with noisy observations. The separate modeling of process and observation noise allows the estimator to appropriately weight the relative reliability of the model versus the sensor measurements, making it effective for tracking and control applications where both system dynamics and measurement quality must be carefully considered (e.g., medical procedure and patient monitoring and modelling, as discussed herein).

Accordingly, in some embodiments, such state-space models can provide a powerful framework for analyzing and designing control systems, particularly in the context of linear systems theory, estimation (such as Kalman filtering), and optimal control. The model's flexibility allows it to be applied to various disciplines, including electrical engineering, robotics, economics, and beyond, providing a unified approach to modeling dynamic systems in both continuous and discrete-time domains.

In Step 1008, based on the output from Steps 1004 and 1006, engine 200 can compile the M1 index. Such compilation can be performed in a similar manner as discussed above at least in relation to Process 300. Accordingly, a level of consciousness for a patient can be computed into a value that can be managed, visualized, shared and/or manipulated to ensure they safe and healthy brain functions are maintained during (and/or after) the procedure when they are subject to anesthesia. As above, an example can be depicted in FIG. 10B.

Accordingly, in Step 1010, engine 200 can intraoperatively manage the EEG signals of the patient based on the M1 index. In some embodiments, operations of Step 1010 can involve input and/or feedback provided by an anesthesiologist, anesthesia caregiver, doctor, and/or any other medical professional that may be involved in the process of anesthesia administration during a medical procedure. Thus, should values dip, move or change to values that are not within a predefined (or preferred) range for the patient (e.g., based on demographics, patterns, the type of procedure, and the like, for example), engine 200 can cause modifications to the anesthesia values to manipulate the desired modifications to the M1 index, as in Step 1012. Such modifications can be performed in a similar manner as discussed above in relation to Process 300 (e.g., as in FIG. 3B), discussed supra, such that, for example, as EEG signal values vary, amounts, quantities and/or types of anesthesia can be modified to curate the desired EEG signal values for the patient, as guided by the M1 index's dynamic application. And, in some embodiments, modifications to M1 index can occur, which can cause the EEG management to change as well, as discussed as well.

And, in Step 1014, engine 200 can continue monitoring the patient, which can be until the procedure is ended and/or patient safely awakes from sedation. Such monitoring can be performed via similar steps of Process 1000 discussed above, which can be performed in a similar manner as discussed above in relation to Process 300. Thus, such monitoring, as discussed above, can ensure a dynamic application of the M1 index to ensure the proper amount and/or type of anesthesia is provided to the patient to ensure a healthy intraoperative and post operative experience

By way of background, advanced surgical systems include many different types of equipment to monitor and anesthetize a patient, assist the surgeon in performing surgical tasks, and maintain the environment of the operating room.

For example, an anesthesiology machine refers to a machine that is used to generate and mix medical gases like oxygen or air and anesthetic agents to induce and maintain anesthesia in patients. Anesthesiology machines deliver oxygen and anesthetic gas to the patient as well as filter out expiratory carbon dioxide. Anesthesia machines may perform following functions provides O2, accurately mix anesthetic gases and vapors, enable patient ventilation, and minimize anesthesia related risks to patients and staff. Anesthesia machines may consist of the following essential components a source of oxygen (O2), O2 flowmeter, vaporizer (anesthetics include isoflurane, halothane, enflurane, desflurane, sevoflurane, and methoxyflurane), patient breathing circuit (tubing, connectors, and valves), scavenging system (removes any excess anesthetics gases). Anesthesia machines may be divided into three parts: the high-pressure system, the intermediate pressure system, and the low-pressure system. The process of anesthesia starts with oxygen flow from pipeline or cylinder through the flowmeter, O2 flows through the vaporizer and picks up the anesthetic vapors, the O2-anesthetic mix then flows through the breathing circuit and into the patient's lungs, usually by spontaneous ventilation or normal respiration. The O2-anesthetic mix then flows through the breathing circuit and into the patient's lungs, usually by spontaneous ventilation or normal respiration. According to some embodiments, an anesthesiology machine can be utilized via the disclosed framework, discussed supra.

A vital signs monitor refers to medical diagnostic instruments and in particular to a portable, battery powered, multi-parametric, vital signs monitoring device that can be used for both ambulatory and transport applications, as well as bedside monitoring. These devices can be used with an isolated data link to an interconnected portable computer allowing snapshot and trended data from the monitoring device to be printed automatically and also allowing default configuration settings to be downloaded to the monitoring device. The monitoring device is capable of use as a stand-alone unit as well as part of a bi-directional wireless communications network that includes at least one remote monitoring station. A number of vital signs monitoring devices are known that are capable of measuring multiple physiologic parameters of a patient, where various sensor output signals are transmitted either wirelessly or by means of a wired connection to at least one remote site, such as a central monitoring station. According to some embodiments, a vital signs monitor can be integrated into disclosed embodiments in a variety of manners, as evident from the below discussion.

A heart rate monitor refers to the sensor(s) and/or sensor system(s) that can be applied in the context of monitoring heart rates. Embodiments are intended to measure, directly or indirectly, any physiological condition from which any relevant aspect of heart rate can be gleaned. For example, some of the embodiments measure different or overlapping physiological conditions to measure the same aspect of heart rate. Alternatively, some embodiments measure the same, different, or overlapping physiological conditions to measure different aspects of heart rate, e.g., number of beats, strength of beats, regularity of beats, beat anomalies, and the like. According to some embodiments, a heart rate monitor can be integrated into disclosed embodiments in a variety of manners (e.g., implemented via sensors 110, as discussed supra).

A pulse oximeter or SpO2 Monitor refers to a plethysmograph or any instrument that measures variations in the size of an organ or body part on the basis of the amount of blood passing through or present in the part. An oximeter is a type of plethysmograph that determines the oxygen saturation of the blood. One common type of oximeter is a pulse oximeter. A pulse oximeter is a medical device that indirectly measures the oxygen saturation of a patient's blood (as opposed to measuring oxygen saturation directly through a blood sample) and changes in blood volume in the skin. A pulse oximeter may include a light sensor that is placed at a site on a patient, usually a fingertip, toe, forehead, or earlobe, or in the case of a neonate, across a foot. Light, which may be produced by a light source integrated into the pulse oximeter, containing both red and infrared wavelengths is directed onto the skin of the patient and the light that passes through the skin is detected by the sensor. The intensity of light in each wavelength is measured by the sensor over time. The graph of light intensity versus time is referred to as the photoplethysmogram (PPG) or, more commonly, simply as the “pleth.” From the waveform of the PPG, it is possible to identify the pulse rate of the patient and when each individual pulse occurs. In addition, by comparing the intensities of two wavelengths when a pulse occurs, it is possible to determine blood oxygen saturation of hemoglobin in arterial blood. This relies on the observation that highly oxygenated blood will relatively absorb more red light and less infrared light than blood with a lower oxygen saturation. According to some embodiments, a pulse oximeter can be integrated into disclosed embodiments in a variety of manners (e.g., implemented via sensors 110, as discussed supra).

An end Tidal CO2 monitor or capnography monitor refers to an instrument which is used for measurement of level of carbon dioxide (referred to as end tidal carbon dioxide, ETCO2) that is released at the end of an exhaled breath. End tidal CO2 monitor or capnography monitor is widely used in anesthesia and intensive care. ETCO2 can be calculated by plotting expiratory CO2 with time. Further, ETCO2 monitor plays a very crucial role for the measurement of applications such as cardiopulmonary resuscitation (CPR), airway assessment, procedural sedation and analgesia, pulmonary diseases such as obstructive pulmonary disease, pulmonary embolism, and the like, heart failure, metabolic disorders, and the like. The instrument can be configured as side stream (diverting) or mainstream (non-diverting). Diverting device transports a portion of a patient's respired gases from the sampling site to the sensor while non-diverting device does not transport gas away. Also, measurement by the instrument is based on the absorption of infrared light by carbon dioxide, where exhaled gas passes through a sampling chamber containing an infrared light source and photodetector on both sides. Based on the amount of infrared light reaching the photodetector, the amount of carbon dioxide present in the gas can be calculated. According to some embodiments, an ETCO2 monitor or capnography monitor can be integrated into disclosed embodiments in a variety of manners (e.g., implemented via sensors 110, as discussed supra).

A blood pressure monitor refers to any instrument that measures blood pressure, particularly in arteries. Blood pressure monitors use a non-invasive technique (by external cuff application) or an invasive technique (by a cannula needle inserted in an artery, used in an operating theater) for measurement, with non-invasive measurement being widely used. The non-invasive method (referred to as sphygmomanometer further) works by measurement of force exerted against arterial walls during ventricular systole (e.g., systolic blood pressure, occurs when heart beats and pushes blood through the arteries) and ventricular diastole (e.g., diastolic blood pressure, occurs when heart rests and is filling with blood) thereby measuring systole and diastole, respectively. It can be of three types, automatic/digital, manual (aneroid-dial), and manual (mercury-column). The sphygmomanometer may include a bladder, a cuff, a pressure meter, a stethoscope, a valve, and a bulb. The cuff then inflates until it fits tightly around your arm, cutting off your blood flow, and then the valve opens to deflate it. It operates by inflating a cuff tightly around the arm, as the cuff reaches the systolic pressure, blood begins to flow around your artery, and creating a vibration which is detected by the meter, which records your systolic pressure. This systolic pressure is recorded. The techniques used for measurement may be, for example: auscultatory or oscillometric. According to some embodiments, a blood pressure monitor can be integrated into disclosed embodiments in a variety of manners (e.g., implemented via sensors 110, as discussed supra).

A body temperature monitor refers to any instrument which is used for measurement of body temperature. The instrument can measure the temperature invasively or non-invasively by placement of sensor into organs such as bladder, rectum, esophagus, tympanum, esophagus, and the like, and mouth, rectum, armpit, and the like, respectively. The sensors are of two types: contact and non-contact. It can be measured in two forms: core temperature and peripheral temperature. Temperature measurement can be done by these sensing technologies: thermocouples, resistive temperature devices (RTDs, thermistors), infrared radiators, bimetallic devices, liquid expansion devices, molecular change-of-state, and silicon diodes. A thermometer which is a commonly used instrument for the measurement of temperature consists of a temperature sensing element (e.g., temperature sensor) and a means for converting to a numerical value. According to some embodiments, a blood temperature monitor can be integrated into disclosed embodiments in a variety of manners (e.g., implemented via sensors 110, as discussed supra).

Respiration rate or breathing rate is the rate at which breathing occurs and is measured by a number of breaths a person takes per minute. The rate is usually measured when a person is at rest and simply involves counting the number of breaths for one minute by counting how many times the chest rises. Normal respiration rates for an adult person at rest are in the range: 12 to 16 breaths per minute. A variation can be an indication of an abnormality/medical condition or a patient's demographic parameters. Hypoxia is a condition with low levels of oxygen in the cells and hypercapnia is a condition in which high levels of carbon dioxide in the bloodstream. Pulmonary disorders, asthma, anxiety, pneumonia, heart diseases, dehydration, drug overdose are some of the abnormal conditions which can bring a change to the respiration rate, thereby increasing or reducing the respiration rate from normal levels. According to some embodiments, monitoring, identification and/or determination of a patient's respiratory rate can be utilized via the disclosed framework, discussed supra.

An electrocardiogram (abbreviated as EKG or ECG, interchangeably) refers to a representation of the electrical activity of the heart (graphical trace of voltage versus time) which is done by placement of electrodes on skin/body surface. The electrodes capture the electrical impulse which travels through the heart causing systole and diastole or the pumping of the heart. This impulse gives a lot of information related to the normal functioning of the heart and the production of impulses. A change may occur due to medical conditions such as arrhythmias (tachycardia where the heart rate becomes faster and bradycardia where the heart rate becomes slower), coronary heart disease, heart attacks, cardiomyopathy. The instrument used for the measurement of the electrocardiogram is called an electrocardiograph which measures the electrical impulses by the placement of electrodes on the surface of the body and represents the ECG by a PQRST waveform. PQRST wave is read as: P wave which represents the depolarization of the left and right atrium and corresponding to atrial contraction, QRS complex indicates ventricular depolarization and represents the electrical impulse as it spreads through the ventricles; T wave indicates ventricular repolarization and follows the QRS complex. According to some embodiments, an electrocardiogram can be utilized via the disclosed framework, discussed supra.

Neuromonitoring, which may also be referred to as Intraoperative neurophysiological monitoring (IONM) refers to an assessment of functions and changes in the brain, brainstem, spinal cord, cranial nerves, and peripheral nerves during a surgical procedure on these organs. It includes both continuous monitoring of neural tissue as well as the localization of vital neural structures. IONM measures changes in these organs which are indicative of irreversible damage, injuries in the organs, aiming at reducing the risk of neurological deficits after operations involving the nervous system. This has also been found to be effective in localization of anatomical structures, including peripheral nerves and sensorimotor cortex, which help in guiding the surgeon during dissection. Electrophysiological modalities which are employed in neuromonitoring are an extracellular single unit and local field recordings (LFP), somatosensory evoked potential (SSEP), transcranial electrical motor evoked potentials (TCeMEP), electromyography (EMG), electroencephalography (EEG), and auditory brainstem response (ABR). The use of neurophysiological monitoring during surgical procedures requires specific anesthesia techniques to avoid interference and signal alteration due to anesthesia. According to some embodiments, neuromonitoring can be utilized via the disclosed framework, discussed supra.

Motor evoked potential (MEP) refers to electrical signals which are recorded from descending motor pathways or muscles following stimulation of motor pathways within the brain. MEP may be calculated by measurement of the action potential which is elicited by non-invasive stimulation of the motor cortex through the scalp. MEP is a widely used technique for intraoperative monitoring and neurophysiological testing of the motor pathways specifically during spinal procedures. The technique of monitoring for measurement of MEP can be defined based on some of the parameters like a site of stimulation (motor cortex or spinal cord), method of stimulation (electrical potential or magnetic field), and site of recording (spinal cord or peripheral mixed nerve and muscle). The target site may be stimulated by the use of electrical or magnetic means. According to some embodiments, MEP can be utilized via the disclosed framework, discussed supra.

Somatosensory evoked potential (which may be abbreviated as SSEP or SEP, interchangeably) refers to the electrical signals which are elicited by the brain and the spinal cord in response to sensory stimulus or touch. SSEP is one of the most frequently used techniques for intraoperative neurophysiological monitoring in spinal surgeries. The method proves to be very reliable which allows for continuous monitoring during a surgical procedure. However, accuracy may be a concern at times in measurement. The sensor stimulus which is commonly given to the organs may be auditory, visual, or somatosensory SEPs and applied on the skin, peripheral nerves of the upper limb, lower limb, or scalp. The stimulation technique may be mechanical (widely used), or electrical (found to give larger and more robust responses), intraoperative spinal monitoring modality. According to some embodiments, somatosensory evoked potential can be utilized via the disclosed framework, discussed supra.

Electromyography (EMG) refers to the evaluation and recording of electrical signals or electrical activity of the skeletal muscles. Electromyography instrument or electromyograph or electromyogram, the instrument for the measurement of the EMG activity works on a technique used for a recording of electrical activity produced by skeletal muscles and evaluation of the functional integrity of individual nerves. The nerves which are monitored by the EMG instrument may be intracranial, spinal, or peripheral nerves. The electrodes which may be used for the acquisition of signals may be invasive and non-invasive electrodes. The technique used for measurement may be spontaneous or triggered. Spontaneous EMG refers to the recording of myoelectric signals during surgical manipulation such as compression, stretching, or pulling of nerves produced; and does not perform external stimulation. Spontaneous EMG may be recorded by the insertion of a needle electrode. Triggered EMG refers to the recording of myoelectric signals during stimulation of target site such as pedicle screws with incremental current intensities. According to some embodiments, electromyography can be utilized via the disclosed framework, discussed supra.

Electroencephalography (EEG) refers to the electrical signals in the brain. Brain cells communicate with each other through electrical impulses. EEG can be used to help detect potential problems associated with this activity. An electroencephalograph is used for the measurement of EEG activity. Electrodes ranging from 8 to 16 pairs are attached to the scalp where each pair of electrodes transmit a signal to one or more recording channels. It is one of the oldest and most commonly utilized modalities for intraoperative neurophysiological monitoring and assessing cortical perfusion and oxygenation during a variety of vascular, cardiac, and neurosurgical procedures. The waves produced by EEG are alpha, beta, theta, and slow/delta. According to some embodiments, electroencephalography can be utilized via the disclosed framework, discussed supra.

Medical visualization systems refer to visualization systems that are used for visualization and analysis of objects (preferably three-dimensional (3D) objects). Medical visualization systems include the selection of points at surfaces, selection of a region of interest, selection of objects. Medical visualization systems may be used for applications diagnosis, treatment planning, intraoperative support, documentation, educational purpose. Medical visualization systems may consist of microscopes, endoscopes/arthroscopes/laparoscopes, fiber optics, surgical lights, high-definition monitors, operating room cameras, and the like. 3D visualization software provides visual representations of scanned body parts via virtual models, offering significant depth and nuance to static two-dimensional medical images. The software facilitates improved diagnoses, narrowed surgical operation learning curves, reduced operational costs, and shortened image acquisition times. According to some embodiments, medical visualization systems can be integrated and/or utilized via the disclosed framework, discussed supra.

Sensors (e.g., sensors 110) are used in association with minimally invasive surgery devices. The sensor may be used in minimally invasive surgeries for tactile sensing of tool—tissue interaction forces. During minimally invasive surgeries field of view and workspace of tools are compromised due to the indirect access to the anatomy and lack of surgeon's hand-eye coordination. The sensors provide a tactile sensation to the surgeon by providing information of shape, stiffness, and texture of organ or tissue (different characteristics) to surgeon's hands through a sense of touch. This detection of a tumor through palpation, which exhibit a ‘tougher’ feel than healthy soft tissue, pulse felt from blood vessels, and abnormal lesions. The sensors may provide in output shape, size, pressure, softness, composition, temperature, vibration, shear, and normal forces. Sensor may be electrical or optical, consisting of capacitive, inductive, piezoelectric, piezoresistive, magnetic, and auditory. The sensors may be used in robotic, laparoscopic, palpation, biopsy, heart ablation, and valvuloplasty. According to some embodiments, a sensor(s) can be utilized via the disclosed framework, discussed supra (e.g., sensors 110).

According to some embodiments, equipment refers to a set of articles, tools, or objects which help to implement or achieve an operation or activity. A medical equipment (or medical imaging equipment) refers to an article, instrument, apparatus, or machine used for diagnosis, prevention, or treatment of a medical condition or disease or detection, measurement, restoration, correction, or modification of structure/function of the body for some health purpose. The medical equipment may perform functions invasively or non-invasively. The medical equipment may consist of components sensor/transducer, signal conditioner, display, data storage unit, and the like. The medical equipment works by taking a signal from a measurand/patient, a transducer for converting one form of energy to electrical energy, signal conditioner such as an amplifier, filters, and the like, to convert the output from the transducer into an electrical value, display to provide a visual representation of measured parameter or quantity, a storage system to store data which can be used for future reference. A medical equipment may perform any function of diagnosis or provide therapy, for example, the equipment delivers air/breaths into the lungs and moves it out of the lungs and out of lungs, to a patient who is physically unable to breathe, or breaths insufficiently. According to some embodiments, medical equipment can be utilized via the disclosed framework, discussed supra.

Robotic systems refer to systems that provide intelligent services and information by interacting with their environment, including human beings, via the use of various sensors, actuators, and human interfaces. These are employed for automating processes in a wide range of applications, ranging from industrial (manufacturing), domestic, medical, service, military, entertainment, space, and the like. The adoption of robotic systems provides several benefits, including efficiency and speed improvements, lower costs, and higher accuracy. Performing medical procedures with the assistance of robotic technology are referred to as medical robotic systems. The medical robotic system market can be segmented by product type into surgical robotic systems, rehabilitative robotic systems, non-invasive radiosurgery robots, hospital & pharmacy robotic systems. Robotic technologies have offered valuable enhancements to medical or surgical processes through improved precision, stability, and dexterity. Robots in medicine help by relieving medical personnel from routine tasks, and by making medical procedures safer and less costly for patients. They can also perform accurate surgery in tiny places and transport dangerous substances. Robotic surgeries are performed using tele-manipulators, which use the surgeon's actions on one side to control the “effector” on the other side. A medical robotic system ensures precision and may be used for remotely controlled, minimally invasive procedures. The systems include computer-controlled electromechanical devices that work in response to controls manipulated by the surgeons. According to some embodiments, robotic systems can be utilized via the disclosed framework, discussed supra.

An electronic health record (EHR) refers to a digital record of a patient's health information, which may be collected and stored systematically over time. It is an all-inclusive patient record and could include demographics, medical history, history of present illness (HPI), progress notes, problems, medications, vital signs, immunizations, laboratory data, and radiology reports. A computer software is used to capture, store, and share patient data in a structured way. The EHR may be created and managed by authorized providers and can make health information instantly accessible to authorized providers across practices and health organizations-such as laboratories, specialists, medical imaging facilities, pharmacies, emergency facilities, and the like. The timely availability of EHR data can enable healthcare providers to make more accurate decisions and provide better care to the patients by effective diagnosis and reduced medical errors. Besides providing opportunities to enhance patient care, it may also be used to facilitate clinical research by combining all patients' demographics into a large pool. For example, the EHR data can support a wide range of epidemiological research on the natural history of disease, drug utilization, and safety, as well as health services research. According to some embodiments, EHRs can be utilized via the disclosed framework, discussed supra.

Equipment tracking systems, such as radio-frequency identification (RFID), for example, refers to a system that tags an instrument with an electronic tag and tracks it using the tag. Typically, this could involve a centralized platform that provides details such as location, owner, contract, and maintenance history for all equipment in real-time. A variety of techniques can be used to track physical assets, including RFID, global positioning system (GPS), Bluetooth low energy (BLE), barcodes, near-field communication NFC, Wi-Fi, and the like. The equipment tracking system include the hardware components, such as RFID tags, GPS trackers, barcodes, and QR codes. The hardware component is placed on the asset, and it communicates with the software (directly or via a scanner), providing it with data about the asset's location and properties. An equipment tracking system uses electromagnetic fields to transmit data from an RFID tag to a reader. Reading of RFID tags may be done by portable or mounted RFID readers. RFID may be very short for low frequency or high frequency for ultra-high frequency. Managing and locating important assets is a key challenge for tracking medical equipment. Time spent searching for critical equipment can lead to expensive delays or downtime, missed deadlines and customer commitments, and wasted labor. The problem has been solved by the use of barcode labels or using manual serial numbers and spreadsheets; however, these require manual labor. The RFID tag may be passive (smaller and less expensive, read ranges are shorter, have no power of their own, and are powered by the radio frequency energy transmitted from RFID readers/antennas) or active (larger and more expensive, read ranges are longer, have a built-in power source and transmitter of their own). Equipment tracking systems may offer advantages, no line of sight required, read Multiple RFID objects at once, scan at a distance, and flexibility. According to some embodiments, equipment tracking systems can be utilized via the disclosed framework, discussed supra.

Techniques operating according to the principles described herein may be implemented in any suitable manner. Included in the discussion above are a series of flow charts showing the steps and acts of various processes for analyzing movement information to determine a time at which to trigger acquisition of a medical image. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of techniques described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and may result from compiling of other code into machine language code or intermediate code that is interpreted by a framework or virtual machine for execution.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, as a thread of a process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include functions, routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a software package or software program application. These functional facilities may, in alternative embodiments, be adapted to interact with other, unrelated functional facilities and/or processes, to implement a software package or software program application.

Some examples of modules have been described herein, which may be implemented as one or more functional facilities for carrying out one or more tasks. It should be appreciated, though, that the modules and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 806 of FIG. 9 described below (i.e., as a portion of a computing device 600) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1A, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 9 is a schematic diagram illustrating a client device showing an example embodiment of a client device that may be used within the present disclosure. Client device 600 may include many more or less components than those shown in FIG. 9. However, the components shown are sufficient to disclose an illustrative embodiment for implementing the present disclosure. Client device 600 may represent, for example, UE 102 discussed above at least in relation to FIG. 1A.

As shown in the figure, in some embodiments, Client device 600 includes a processing unit (CPU) 622 in communication with a mass memory 630 via a bus 624. Client device 600 also includes a power supply 626, one or more network interfaces 650, an audio interface 652, a display 654, a keypad 656, an illuminator 658, an input/output interface 660, a haptic interface 662, an optional global positioning systems (GPS) receiver 664 and a camera(s) or other optical, thermal or electromagnetic sensors 666. Device 600 can include one camera/sensor 666, or a plurality of cameras/sensors 666, as understood by those of skill in the art. Power supply 626 provides power to Client device 600.

Client device 600 may optionally communicate with a base station (not shown), or directly with another computing device. In some embodiments, network interface 650 is sometimes known as a transceiver, transceiving device, or network interface card (NIC).

Audio interface 652 is arranged to produce and receive audio signals such as the sound of a human voice in some embodiments. Display 654 may be a liquid crystal display (LCD), gas plasma, light emitting diode (LED), or any other type of display used with a computing device. Display 654 may also include a touch sensitive screen arranged to receive input from an object such as a stylus or a digit from a human hand.

Keypad 656 may include any input device arranged to receive input from a user. Illuminator 658 may provide a status indication and/or provide light.

Client device 600 also includes input/output interface 660 for communicating with external. Input/output interface 660 can utilize one or more communication technologies, such as USB, infrared, Bluetooth™, or the like in some embodiments. Haptic interface 662 is arranged to provide tactile feedback to a user of the client device.

Optional GPS transceiver 664 can determine the physical coordinates of Client device 600 on the surface of the Earth, which typically outputs a location as latitude and longitude values. GPS transceiver 664 can also employ other geo-positioning mechanisms, including, but not limited to, triangulation, assisted GPS (AGPS), E-OTD, CI, SAI, ETA, BSS or the like, to further determine the physical location of client device 600 on the surface of the Earth. In one embodiment, however, Client device 600 may through other components, provide other information that may be employed to determine a physical location of the device, including for example, a MAC address, Internet Protocol (IP) address, or the like.

Mass memory 630 includes a RAM 632, a ROM 634, and other storage means. Mass memory 630 illustrates another example of computer storage media for storage of information such as computer readable instructions, data structures, program modules or other data. Mass memory 630 stores a basic input/output system (“BIOS”) 640 for controlling low-level operation of Client device 600. The mass memory also stores an operating system 641 for controlling the operation of Client device 600.

Memory 630 further includes one or more data stores, which can be utilized by Client device 600 to store, among other things, applications 642 and/or other information or data. For example, data stores may be employed to store information that describes various capabilities of Client device 600. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 600.

Applications 642 may include computer executable instructions which, when executed by Client device 600, transmit, receive, and/or otherwise process audio, video, images, and enable telecommunication with a server and/or another user of another client device. Applications 642 may further include a client that is configured to send, to receive, and/or to otherwise process gaming, goods/services and/or other forms of data, messages and content hosted and provided by the platform associated with engine 200 and its affiliates.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A method comprising:
receiving, by an application, patient-specific data including at least one of demographic, clinical or procedural information;
determining, by the application, electroencephalogram (EEG) signals for the patient;
executing, by the application, a state-space model on the EEG signals to generate transformed signal data;
compiling, by the application, a consciousness index (M1 index) based on the transformed signal data;
managing, by the application, EEG signal data in real time during a medical procedure using the compiled M1 index;
managing, by the application, anesthesia levels based on the M1 index and the managed EEG signal data; and
continuing, by the application, patient monitoring until a condition selected from the group consisting of (i) completion of the medical procedure and (ii) safe return to consciousness is satisfied.

2. The method of claim 1, wherein compiling the M1 index includes applying a classifier that accounts for age-dependent neural dynamics, including characteristics present in older adults and individuals with neurodegenerative conditions such as undiagnosed dementia.

3. The method of claim 1, further comprising preprocessing the EEG signal data to remove electromyographic (EMG) artifacts prior to executing the state-space model.

4. The method of claim 1, wherein managing EEG signal data includes detecting deviation from a predefined M1 threshold indicative of anesthetic depth and adjusting signal interpretation accordingly.

5. The method of claim 1, further comprising comparing the M1 index over time to determine a risk level for postoperative delirium associated with the depth and duration of anesthesia-induced unconsciousness.

6. The method of claim 5, wherein determining the delirium risk comprises generating an alert or recommendation when the M1 index remains below a cognitive suppression threshold for a predefined time interval.

7. The method of claim 1, wherein the state-space model comprises a dynamic Bayesian model that captures transitions between different neural states associated with varying levels of consciousness.

8. The method of claim 1, wherein managing anesthesia levels includes automatic adjustment of an anesthetic agent infusion rate based on real-time updates to the M1 index.

9. A system comprising:
a processor configured to:
receive, by an application, patient-specific data including at least one of demographic, clinical or procedural information;
determine, by the application, electroencephalogram (EEG) signals for the patient;
execute, by the application, a state-space model on the EEG signals to generate transformed signal data;
compile, by the application, a consciousness index (M1 index) based on the transformed signal data;
manage, by the application, EEG signal data in real time during a medical procedure using the compiled M1 index;
manage, by the application, anesthesia levels based on the M1 index and the managed EEG signal data; and
continue, by the application, patient monitoring until a condition selected from the group consisting of (i) completion of the medical procedure and (ii) safe return to consciousness is satisfied.

10. The system of claim 9, wherein compiling the M1 index includes applying a classifier that accounts for age-dependent neural dynamics, including characteristics present in older adults and individuals with neurodegenerative conditions such as undiagnosed dementia.

11. The system of claim 9, further comprising preprocessing the EEG signal data to remove electromyographic (EMG) artifacts prior to executing the state-space model.

12. The system of claim 9, wherein managing EEG signal data includes detecting deviation from a predefined M1 threshold indicative of anesthetic depth and adjusting signal interpretation accordingly.

13. The system of claim 9, further comprising comparing the M1 index over time to determine a risk level for postoperative delirium associated with the depth and duration of anesthesia-induced unconsciousness.

14. The system of claim 13, wherein determining the delirium risk comprises generating an alert or recommendation when the M1 index remains below a cognitive suppression threshold for a predefined time interval.

15. The system of claim 9, wherein the state-space model comprises a dynamic Bayesian model that captures transitions between different neural states associated with varying levels of consciousness.

16. The system of claim 9, wherein managing anesthesia levels includes automatic adjustment of an anesthetic agent infusion rate based on real-time updates to the M1 index.

17. A non-transitory computer-readable storage medium tangibly encoded with computer-executable instructions, that when executed by a processor, perform a method comprising:
receiving, by an application, patient-specific data including at least one of demographic, clinical or procedural information;
determining, by the application, electroencephalogram (EEG) signals for the patient;
executing, by the application, a state-space model on the EEG signals to generate transformed signal data;
compiling, by the application, a consciousness index (M1 index) based on the transformed signal data;
managing, by the application, EEG signal data in real time during a medical procedure using the compiled M1 index;
managing, by the application, anesthesia levels based on the M1 index and the managed EEG signal data; and
continuing, by the application, patient monitoring until a condition selected from the group consisting of (i) completion of the medical procedure and (ii) safe return to consciousness is satisfied.

18. The non-transitory computer-readable storage medium of claim 17, wherein compiling the M1 index includes applying a classifier that accounts for age-dependent neural dynamics, including characteristics present in older adults and individuals with neurodegenerative conditions such as undiagnosed dementia.

19. The non-transitory computer-readable storage medium of claim 17, further comprising preprocessing the EEG signal data to remove electromyographic (EMG) artifacts prior to executing the state-space model.

20. The non-transitory computer-readable storage medium of claim 17, wherein managing EEG signal data includes detecting deviation from a predefined M1 threshold indicative of anesthetic depth and adjusting signal interpretation accordingly.
